(19) United States
(12) Patent Application Publication
(10) Pub. No.: US 2008/0318974 A1
(43) Pub. Date: Dec. 25, 2008

(54) Compounds Useful for Inhibiting Chk1

(75) Inventors: John Joseph Gaudino, Longmont, CO (US); Francine S. Farouz, Mercer Island, WA (US); Ryan Coatsworth Holcomb, Salt Lake City, UT (US); Eugene D. Thorsett, Half Moon Bay, CA (US)

Correspondence Address: ELI LILLY & COMPANY, PATENT DIVISION, P.O. BOX 6288, INDIANAPOLIS, IN, 46206-6288, US

(73) Assignee: Icos Corporation, Bothell, WA (US)

(21) Appl. No.: 11/659,389

(22) PCT Filed: Aug. 18, 2005
(86) PCT No.: PCT/US05/29518

§ 371 Date: Oct. 24, 2007

Related U.S. Application Data
(60) Provisional application No. 60/602,968, filed on Aug. 19, 2004.

(51) Int. Cl. A61K 31/497; C07D 401/12; A61P 35/00; C07D 401/04

(52) U.S. Cl. 514/255.05; 544/336; 544/405

(57) ABSTRACT

Aryl- and heteroaryl-substituted urea compounds useful in the treatment of diseases and conditions related to DNA damage or lesions in DNA replication are disclosed. Methods of making the compounds, and their use as therapeutic agents, for example, in treating cancer and other diseases characterized by defects in DNA replication, chromosome segregation, or cell division also are disclosed.

### FIELD OF THE INVENTION

The present invention relates to compounds useful for inhibiting enzymes that maintain and repair the integrity of genetic material. More particularly, the present invention relates to a series of aryl- and heteroaryl-substituted urea compounds, methods of making the compounds, and their use as therapeutic agents, for example, in treating cancer and other diseases characterized by defects in deoxyribonucleic acid (DNA) replication, chromosome segregation, or cell division.

### BACKGROUND OF THE INVENTION

A large variety of diseases, conditions, and disorders (hereinafter “indications”) are characterized as involving aberrantly proliferating cells. As used herein, “aberrantly proliferating cells” (or “aberrant cell proliferation”) means cell proliferation that deviates from the normal, proper, or expected course. For example, aberrant cell proliferation includes inappropriate proliferation of cells wherein DNA or other cellular components have become damaged or defective. Aberrant cell proliferation also includes indications caused by, mediated by, or resulting in inappropriately high levels of cell division, inappropriately low levels of cell death (e.g., apoptosis), or both. Such indications can be characterized, for example, by single or multiple local abnormal proliferations of cells, groups of cells or tissue(s), and include cancerous (benign or malignant) and noncancerous indications.

By definition, all cancers (benign and malignant) involve some form of aberrant cell proliferation. Nonlimiting examples include carcinomas and sarcomas. Others are discussed below. Some noncancerous indications also involve aberrant cell proliferation. Nonlimiting examples of noncancerous indications involving aberrant cell proliferation include rheumatoid arthritis, psoriasis, vitiligo, Wegener's granulomatosis, and systemic lupus. Others are discussed below.

One approach to treating indications involving aberrantly proliferating cells involves the use of DNA damaging agents. These agents are designed to kill aberrantly proliferating cells by disrupting vital cellular processes such as DNA metabolism, DNA synthesis, DNA transcription, and microtubule spindle formation. They also can operate, for example, by introducing lesions into DNA that perturb chromosomal structural integrity. DNA damaging agents are designed and administered in ways that attempt to induce maximum damage and consequent cell death in aberrantiy proliferating cells with a minimum damage to normal, healthy cells.

A large variety of DNA damaging agents have been developed to date. Others are also in development. DNA damaging agents include chemotherapeutics and radiation. Unfortunately, the effectiveness of DNA damaging agents in treating conditions involving aberrant cell proliferation have been less than desired, particularly in the treatment of cancer. The selectivity of such agents for aberrantly proliferating cells over healthy cells (sometimes referred to as the therapeutic index) often is marginal.

Moreover, all cells have sensing and repair mechanisms that can work at cross purposes to DNA damaging agents. Such sensing mechanisms, called cell cycle checkpoints, help to maintain the order of the various cell replication stages and to ensure that each step is executed with high fidelity (Hartwell et al., Science, 246:629-634 (1989); Weinert et al., Genes Dev., 8:652 (1994)). When cells detect DNA damage, including damage purposefully induced by DNA damaging agents, certain signaling pathways activate cell cycle checkpoints and the cell replication cycle temporarily ceases (“arrests”). This arrest allows time for aberrantly proliferating cells to repair their DNA, often to a degree sufficient to allow the affected cells to continue to survive and proliferate. This unwanted repair tends to undermine efforts to induce DNA damage sufficient to kill aberrantly proliferating cells.

For example, the chemotherapeutic agent called Gemzar™ (gemcitabine, or 2′,2′ difluoro-2′-deoxycytidine) damages DNA by incorporating itself into DNA during synthesis. Left unrepaired, damaged DNA generally is rendered incapable of sustaining life. In many targeted cells, however, cell cycle checkpoints detect the improperly made (or otherwise damaged) DNA. The activated cell cycle checkpoints trigger cell cycle arrest for a time sufficient to allow damaged DNA to be repaired. This is one way in which aberrantly proliferating cells are theorized to resist the cell-killing effect of DNA-damaging agents such as chemotherapeutics, radiation, and other therapies.

Other DNA-damaging agents cause tumor cells to arrest in S-phase. Tumor cells have been observed to resist certain chemotherapeutics simply by arresting in S phase while the chemotherapeutic agent is being administered. Then, as soon as the drug is removed, DNA damage is repaired, cell cycle arrest ceases, and the cells progress through the remainder of the cell cycle (Shi et al., Cancer Res. 61:1065-1072, 2001). Other therapeutics cause cell cycle arrest at other checkpoints, including G1 and G2 (described more fully below). DNA damaging agents that activate cell cycle checkpoints generally are referred to herein as “checkpoint activators.” DNA damaging agents that activate the checkpoint designated “Chk1” (pronounced “check-one”) are referred to herein as “Chk1 activators.” Inhibitors of such checkpoints, generally and specifically, are referred to herein as “checkpoint inhibitors” and “Chk1 inhibitors,” respectively. Inhibition of various DNA damage checkpoints therefore is expected to assist in preventing cells from repairing therapeutically induced DNA damage and to sensitize targeted cells to DNA damaging agents. Such sensitization is in turn expected to increase the therapeutic index of these therapies.

To more fully understand the present invention, the following is a more detailed discussion of cell cycle phases and the role of Chk1.

The cell cycle is structurally and functionally the same in its basic process and mode of regulation across all eukaryotic species. The mitotic (somatic) cell cycle consists of four phases: the G1 (gap) phase, the S (synthesis) phase, the G2 (gap) phase, and the M (mitosis) phase. The G1, S, and G2 phases are collectively referred to as interphase of the cell cycle.

During the G1 phase, biosynthetic activities of the cell progress at a high rate. The S phase begins when DNA synthesis starts, and ends when the DNA content of the nucleus of the cell has been replicated and two identical sets of chromosomes are formed.

The cell then enters the G2 phase, which continues until mitosis starts. In mitosis, the chromosomes pair and separate, two new nuclei form, and cytokinesis occurs in which the cell splits into two daughter cells each receiving one nucleus containing one of the two sets of chromosomes. Cytokinesis terminates the M phase and marks the beginning of interphase of the next cell cycle. The sequence in which cell cycle events proceed is tightly regulated, such that the initiation of one cell cycle event is dependent on the completion of the prior cell cycle event. This allows fidelity in the duplication and segregation of genetic material from one generation of somatic cells to the next.

It has been reported that cell cycle checkpoints comprise at least three distinct classes of polypeptides, which act sequentially in response to cell cycle signals or defects in chromosomal mechanisms (Carr, A. M., Science, 271:314-315 (1996)). The first class is a family of proteins that detect or sense DNA damage or abnormalities in the cell cycle. These sensors include Ataxia-telangiectasia Mutated protein (Atm) and Ataxia-telangiectasia Rad-related protein (Atr). The second class of polypeptides amplify and transmit the signal detected by the detector and is exemplified by Rad53 (Alen et al. Genes Dev. 8:2416-2488 (1994)) and Chk1. A third class of polypeptides includes cell cycle effectors, such as p53, which mediate a cellular response, for example, arrest of mitosis and apoptosis.

Much of the current understanding of the function of cell cycle checkpoints has been derived from the study of tumor derived cell lines. In many cases, tumor cells have lost key cell cycle check points (Hartwell et al., Science 266:1821-28, 1994). It has been reported that a key step in the evolution of cells to a neoplastic state is the acquisition of mutations that inactivate cell cycle checkpoint pathways, such as those involving p53 (Weinberg, R. A., Cell 81:323 330, 1995; Levine, A. J., Cell 88:3234 331, 1997). Loss of these cell cycle checkpoints results in the replication of tumor cells despite DNA damage.

Noncancerous tissue, which has intact cell cycle checkpoints, typically is insulated from temporary disruption of a single checkpoint pathway. Tumor cells, however, have defects in pathways controlling cell cycle progression such that the perturbation of additional checkpoints renders them particularly sensitive to DNA damaging agents. For example, tumor cells that contain mutant p53 are defective both in the G1 DNA damage checkpoint and in the ability to maintain the G2 DNA damage checkpoint (Bunz et al., Science, 282:1497-1501, 1998). Checkpoint inhibitors that target initiation of the G2 checkpoint or the S phase checkpoint are expected to further cripple the ability of these tumor cells to repair DNA damage and, therefore, are candidates to enhance the therapeutic index of both radiation and systemic chemotherapy (Gesner, T., Abstract at SRI Conference: Protein Phosphorylation and Drug Discovery World Summit, March 2003).

In the presence of DNA damage or any impediment to DNA replication, the checkpoint proteins Atm and Atr initiate a signal transduction pathway leading to cell cycle arrest. Atm has been shown to play a role in a DNA damage checkpoint in response to ionizing radiation. Atr is stimulated by agents that cause double strand DNA breaks, single strand DNA breaks, and agents that block DNA radiation.

Chk1 is a protein kinase that lies downstream from Atm and/or Atr in the DNA damage checkpoint signal transduction pathway (Sanchez et al., Science, 277:1497-1501, 1997; U.S. Pat. No. 6,218,109). In mammalian cells, Chk1 is phosphorylated in response to agents that cause DNA damage including ionizing radiation, ultraviolet (UV) light, and hydroxyurea (Sanchez et al., supra; Lui et al., Genes Dev., 14:1448-1459, 2000). This phosphorylation, which activates Chk1 in mammalian cells, is dependent on Atm (Chen et al., Oncogene, 18:249-256, 1999) and Atr (Lui et al., supra). Furthermore, Chk1 has been shown to phosphorylate both weel (O'Connell et al., EMBO J., 16:545-554, 1997) and Pds1 (Sanchez et al., Science, 286:1166-1171, 1999), gene products known to be important in cell cycle control.

These studies demonstrate that mammalian Chk1 plays a role in the Atm dependent DNA damage checkpoint leading to arrest at S phase. A role for Chk1 in the S phase mammalian cells has recently been elucidated (Feijoo et al., J. Cell. Biol., 154:913-923, 2001; Zhao et al., PNAS USA, 99:14795-800, 2002; Xiao et al., J Biol Chem., 278(24):21767-21773, 2003; Sorensen et al., Cancer Cell, 3(3):247-58, 2003) highlighting the role of Chk1 in monitoring the integrity of DNA synthesis. Chk1 invokes an S-phase arrest by phosphorylating Cdc25A, which regulates cyclinA/cdk2 activity (Xiao et al., supra and Sorensen et al., supra). Chk1 also invokes a G2 arrest by phosphorylating and inactivating Cdc25C, the dual specificity phosphatase that normally dephosphorylates cyclin-B/cdc2 (also known as Cdk1) as cells progress from G2 into mitosis (Fernery et al., Science, 277:1495-7, 1997; Sanchez et al., supra; Matsuoka et al., Science, 282:1893-1897, 1998; and Blasina et al., Curr. Biol., 9:1-10, 1999). In both cases, regulation of Cdk activity induces a cell cycle arrest to prevent cells from entering mitosis in the presence of DNA damage or unreplicated DNA.

Additional classes of cell cycle checkpoint inhibitors operate at either the G1 or G2/M phase. UCN-01, or 7-hydroxystaurosporine, originally was isolated as a nonspecific kinase inhibitor having its primary effect on protein kinase C, but recently it has been found to inhibit the activity of Chk1 and abrogate the G2 cell cycle checkpoint (Shi et al., supra). Thus, UCN-01 is a nonselective Chk1 inhibitor, and is toxic to cells at high doses. At low doses, it nonspecifically inhibits many cellular kinases and also inhibits the G1 checkpoint (Tenzer and Pruschy, Curr. Med. Chem. Anti-Cancer Agents, 3:35-46, 2003).

UCN-01 has been used in conjunction with cancer therapies, such as radiation, the anti-cancer agent camptothecin (Tenzer and Pruschy, supra), and gemcitabine (Shi et al., supra), with limited success. In addition, UCN-01 also has been used to potentiate the effects of temozolomide (TMZ)-induced DNA mismatch repair (MMR) in glioblastoma cells (Hirose et al., Cancer Res., 61:5843-5849, 2001). In the clinic, UCN-01 is not an effective chemotherapeutic as expected, possibly due to a failure in treatment scheduling and a lack of identification of particular key molecular targets (Grant and Roberts, Drug Resistance Updates, 6:15-26, 2003). Thus, Mack et al. report cell cycle-dependent potentiation of cis-platin by UCN-01 in a cultured nonsmall-cell lung carcinoma cell line, but do not identify with specificity the key cell cycle checkpoint(s) targeted by UCN-01. (Mack et al., Cancer Chemother. Pharmacol., 51(4):337-348, 2003).

Several other strategies exist for sensitizing tumor cells to treatment with cell cycle affecting chemotherapeutics. For example, administration of 2-aminopurine abrogates multiple cell cycle checkpoint mechanisms, such as mimosine-induced G1 arrest or hydroxyurea-induced S phase arrest, allowing the cell to progress into and through mitosis (Andreassen et al., Proc Natl Acad Sci USA, 86:2272-2276, 1992). Caffeine, a methylxanthine, has also been used to enhance cytotoxicity of DNA-damaging agents, such as cis-platin and ionizing radiation, by mediating progression through the G2 checkpoint and thereby inducing cell death. (Bracey et al., Clin. Cancer Res., 3:1371-1381, 1997). However, the dose of caffeine used to accomplish the cell cycle abrogation exceeds clinically acceptable levels and is not a viable therapeutic option. Additionally, antisense nucleotides to Chk1 kinase have been used to increase sensitivity to the topoisomerase inhibitor BNP1350 (Yin et al., Biochem. Biophys. Res. Commun., 295:435-44, 2002), but demonstrate problems typically associated with antisense treatment and gene therapy.

Chk1 inhibitors have been disclosed in WO 02/070494, WO 04/014876, and WO 03/101444. Additional Chk1 inhibitors include diarylurea compounds, e.g., aryl- and heteroaryl-substituted urea compounds disclosed in U.S. Patent Publication No. 2003-0069284 A1; methylxanthines and related compounds (Fan et al., Cancer Res., 55:1649-54 (1995); ureidothiophenes (WO 03/029241); N-pyrrolopyridinyl carboxamides (WO 03/28724); antisense Chk1 nucleotides (WO 01/57206); Chk1 receptor antagonists (WO 00/16781); heteroaromatic carboxamide derivatives (wo 03/037886); aminothiophenes (WO 03/029242); (indazolyl)benzimidazoles (WO 03/004488); heterocyclic-hydroxyiminofluorenes (WO 02/16326); scytoneman skeleton-containing derivatives (scytonemin) (U.S. Pat. No. 6,495,586); heteroarylbenzamides (WO 01/53274); indazole compounds (WO 01/53268); indolacarbazoles (see Tenzer et al., supra); chromane derivatives (WO 02/070515); paullones (Schultz et al., J. Med. Chem., 42:2909-2919 (1999)); indenopyrazoles (WO 99/17769); flavones (Sedlacek et al., Int. J. Oncol., 9:1143-1168 (1996); peptide derivatives of peptide loop of serine threonine kinases (WO 98/53050); and oxindoles (WO 03/051838).

However, a need still exists in the art for effective and selective inhibitors of Chk1. The present invention addresses this and other needs.

### SUMMARY OF THE INVENTION

The present invention relates to potent and selective inhibitors of the checkpoint kinase Chk1. The present Chk1 inhibitors are useful in treating indications involving aberrant cell proliferation, and as chemosensitizing and radiosensitizing agents in the treatment of indications related to DNA damage or lesions in DNA replication.

Therefore, one aspect of the present invention is to provide compounds of structural formula (I). The compounds are useful in a method of inhibiting Chk1 comprising a step of administering an effective amount of a compound of structural formula (I) to an individual.

Compounds of formula (I) have a structural formula:

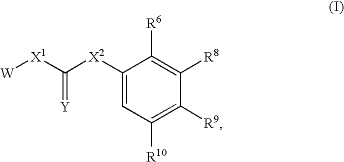

wherein X¹ is null, —O—, —S—, —CH₂—, or —N(R¹)—;

X² is —O—, —S—, or —N(R¹)-;

Y is O or S; or ═Y represents two hydrogen atoms attached to a common carbon atom;

W is selected from the group consisting of heteroaryl, aryl, and C₁₋₆alkyl substituted with a heteroaryl or aryl group, wherein (a) said aryl or heteroaryl group of W is substituted with at least one of CF₃ and heteroaryl, (b) said aryl group of group W is optionally substituted with one to three substituents represented by R², and (c) said heteroaryl group of group W is optionally substituted with one to three substituents represented by R⁵;

R¹ is selected from the group consisting of hydro, C₁₋₆alkyl, C₂₋₆alkenyl, C₂₋₆alkynyl, and aryl;

R² is selected from the group consisting of heteroaryl, halo, optionally substituted C₁₋₆alkyl, C₂₋₆alkenyl, OCF₃, NO₂, CN, NC, N(R³)₂, OR³, CO₂R³, C(O)N—(R³)₂, C(O)R³, N(R¹)COR³, N(R¹)C(O)OR³, N(R¹)C(O)C₁₋₆alkyleneC(O)R³, N(R¹)C(O)C₁₋₆alkyleneC(O)OR³, N(R¹)C(O)C₁₋₆alkyleneOR³, N(R¹)C(O)C₁₋₆alkyleneNHC(O)OR³, N(R¹)C(O)C₁₋₆alkyleneSO₂NR³, C₁₋₆alkyleneOR³, and SR³;

R³ is selected from the group consisting of hydro, halo, C₁₋₆alkyl, C₂₋₆alkenyl, cycloalkyl, aryl, heteroaryl, CO₂R⁴, SO₂R⁴, C₁₋₆alkyl substituted with one or more of halo, hydroxy, aryl, heteroaryl, heterocycloalkyl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF₃, C₁₋₆alkyleneN(R⁴)₃⁺, C₃₋₈heterocycloalkyl, and CH(C₁₋₆alkyleneN(R⁴)₂)₂, or two R³ groups are taken together to form an optionally substituted 3- to 6-membered aliphatic ring;

R⁴ is selected from the group consisting of hydro, C₁₋₆alkyl, cycloalkyl, aryl, heteroaryl, C₁₋₆alkylenearyl, and SO₂C₁₋₆alkyl, or two R⁴ groups are taken together to form an optionally substituted 3- to 6-membered ring;

R⁵ is selected from the group consisting of C₁₋₆alkyl, aryl, heteroaryl, heterocycloalkyl, N(R³)₂, OR³, halo, N₃, CN, C₁₋₆alkylenearyl, C₁₋₆alkyleneN(R³)₂, C(O)R³, C(O)OR³, C(O)N(R³)₂, N(R¹)C(O)R³, N(R¹)C(O)OR³, CF₃, and

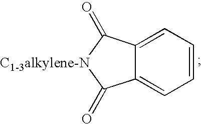

R⁶ is selected from the group consisting of OR¹¹, —C≡C—R⁷, and heteroaryl;

R⁷ is selected from the group consisting of hydro, C₁₋₆alkyl, aryl, C₁₋₆alkylenearyl, heteroaryl, C₁₋₆alkyleneheteroaryl, alkoxy;

R⁸, R⁹, and R¹⁰, independently, are selected from the group consisting of hydro, halo, optionally substituted C₁₋₆alkyl, C₂₋₆alkenyl, C₂₋₆alkynyl, OCF₃, CF₃, NO₂, CN, NC, N(R³)₂, OR³, CO₂R³, C(O)N(R³)₂, C(O)R³, N(R¹)COR³, N(R¹)C(O)OR³, N(R⁸)C(O)OR³, N(R¹)C(O)C₁₋₆alkyleneC(O)R³, N(R¹)C(O)C₁₋₆alkyleneC(O)OR³, N(R¹)C(O)C₁₋₆alkyleneOR³, N(R¹)C(O)C₁₋₆alkyleneNHC(O)OR³, N(R¹)C(O)C₁₋₆alkyleneSO₂NR³, C₁₋₆alkyleneOR³, and SR³;

R¹¹ is selected from the group consisting of hydro, C₁₋₆alkyl, C₂₋₆alkenyl, cycloalkyl, heterocycloalkyl, aryl, heteroaryl, SO₂R⁴, C₁₋₆alkyl substituted with one or more of halo, hydroxy, aryl, heteroaryl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF₃, C₁₋₆alkyleneN(R⁴)₃⁺, C₃₋₈heterocycloalkyl, and CH(C₁₋₆alkyleneN(R⁴)₂)₂;

or a pharmaceutically acceptable salt, or prodrug, or solvate thereof.

Another aspect of the present invention is to provide pharmaceutical compositions comprising one or more compound of structural formula (I), and use of the compositions in a therapeutic treatment of a disease or disorder, wherein inhibition of Chk1, in vivo or ex vivo, provides a therapeutic benefit or is of research or diagnostic interest.

Yet another aspect of the present invention is to provide a method of sensitizing cells in an individual undergoing a chemotherapeutic or radiotherapeutic treatment for a medical condition comprising administration of a compound of structural formula (I) in combination with a chemotherapeutic agent, a radiotherapeutic agent, or both, to the individual. A nonlimiting indication treated by this method is a cancer.

Another aspect of the present invention is to provide a method of inhibiting or preventing aberrant cell proliferation. In one embodiment, a method comprises contacting a cell population comprising aberrantly proliferating cells with at least one Chk1 activator in an amount and for a time sufficient to substantially synchronize cell cycle arrest among the aberrantly proliferating cells. Upon achieving substantial synchronization of cell cycle arrest in the cell population, the cell population is contacted with at least one Chk1 inhibitor in an amount and for a time sufficient to substantially abrogate the cell cycle arrest.

These and other aspects of the present invention will become apparent from the following detailed description of the preferred embodiments.

### DETAILED DESCRIPTION OF THE PREFERRED EMBODIMENTS

Compounds of the present invention have a structural formula (I):

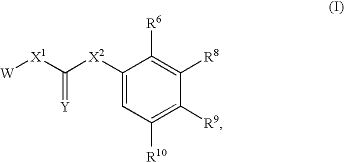

wherein X¹ is null, —O—, —S—, —CH₂—, or —N(R¹);

X² is —O—, —S—, or —N(R¹)—;

Y is O or S; or ═Y represents two hydrogen atoms attached to a common carbon atom;

W is selected from the group consisting of heteroaryl, aryl, and C₁₋₆alkyl substituted with a heteroaryl or aryl group, wherein (a) said aryl or heteroaryl group of group W is substituted with at least one of CF₃ and heteroaryl; (b) said aryl group of group W is optionally substituted with one to three substituents represented by R², and (c) said heteroaryl group of group W is optionally substituted with one to three substituents represented by R⁵;

R¹ is selected from the group consisting of hydro, C₁₋₆alkyl, C₂₋₆alkenyl, C₂₋₆alkynyl, and aryl;

R² is selected from the group consisting of heteroaryl, halo, optionally substituted C₁₋₆alkyl, C₂₋₆alkenyl, OCF₃, NO₂, CN, NC, N(R³)₂, OR³, CO₂R³, C(O)N(R³)₂, C(O)R³, N(R¹)COR³, N(R¹)C(O)OR³, N(R—)C(O)—C₁₋₆alkyleneC(O)R³, N(R¹)C(O)C₁₋₆alkyleneC(O)OR³, N(R¹)—C(O)C₁₋₆alkyleneOR³, N(R¹) C(O)C₁₋₆alkyleneNHC(O)OR³, N(R¹)C(O)C₁₋₆alkyleneSO₂NR³, C₁₋₆alkyleneOR³, and SR³;

R³ is selected from the group consisting of hydro, halo, C₁₋₆alkyl, C₂₋₆alkenyl, cycloalkyl, aryl, heteroaryl, CO₂R⁴, SO₂R⁴, C₁₋₆alkyl substituted with one or more of halo, hydroxy, aryl, heteroaryl, heterocycloalkyl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF₃, C₁₋₆alkyleneN(R⁴)₃′, C₃₋₈heterocycloalkyl, and CH(C₁₋₆alkyleneN(R⁴)₂)₂, or two R³ groups are taken together to form an optionally substituted 3- to 6-membered aliphatic ring;

R⁴ is selected from the group consisting of hydro, C₁₋₆alkyl, cycloalkyl, aryl, heteroaryl, C₁₋₆alkylenearyl, and SO₂C₁₋₆alkyl, or two R⁴ groups are taken together to form an optionally substituted 3- to 6-membered ring;

R⁵ is selected from the group consisting of C₁₋₆alkyl, aryl, heteroaryl, heterocycloalkyl, N(R³)₂, OR³, halo, N₃, CN, C₁₋₆alkylenearyl, C₁₋₆alkyleneN(R³)₂, C(O)R³, C(O)OR³, C(O)N(R³)₂, N(R¹)C(O)R³, N(R¹)C(O)OR³, CF₃, and

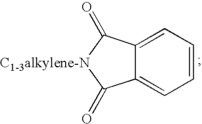

R⁶ is selected from the group consisting of OR¹¹, —C≡C—R⁷, and heteroaryl;

R⁷ is selected from the group consisting of hydro, C₁₋₆alkyl, aryl, C₁₋₆alkylenearyl, heteroaryl, C₁₋₆alkyleneheteroaryl, alkoxy;

R³, R⁹, and R¹⁰, independently, are selected from the group consisting of hydro, halo, optionally substituted C₁₋₆alkyl, C₂₋₆alkenyl, C₂₋₆alkynyl, OCF₃, CF₃, NO₂, CN, NC, N(R³)₂, OR³, CO₂R³, C(O)N(R³)₂, C(O)R³, N(R¹)COR³, N(R¹)C(O)OR³, N(R⁸)C(O)OR³, N(R¹)C(O)C₁₋₆alkyleneC(O)R³, N(R¹)C(O)C₁₋₆alkyleneC(O)OR³, N(R¹)C(O)C₁₋₆alkyleneOR³, N(R¹)C(O)C₁₋₆alkyleneNHC(O)OR³, N(R¹)C(O)C₁₋₆alkyleneSO₂NR³, C₁₋₆alkyleneOR³, and SR³;

R¹¹ is selected from the group consisting of hydro, C₁₋₆alkyl, C₂₋₆alkenyl, cycloalkyl, heterocycloalkyl, aryl, heteroaryl, SO₂R⁴, C₁₋₆alkyl substituted with one or more of halo, hydroxy, aryl, heteroaryl, N(R⁴)₂, and SO₂R⁴, C₁₋₆alkylenearyl, C₁₋₆alkyleneheteroaryl, C₁₋₆alkyleneC₃₋₈heterocycloalkyl, C₁₋₆alkyleneSO₂aryl, optionally substituted C₁₋₆alkyleneN(R⁴)₂, OCF₃, C₁₋₆alkyleneN(R⁴)₃⁺, C₃₋₈heterocycloalkyl, and CH(C₁₋₆alkyleneN(R⁴)₂)₂;

and a pharmaceutically acceptable salt, or prodrug, or solvate thereof.

Preferred compounds of the present invention are those wherein X¹ and X² are —N(H)—;

Y is O or S; and

Preferably W is heteroaryl. In one embodiment, W is heteroaryl containing at least two heteroatoms selected from the group consisting of N, O, and S, said heteroaryl ring optionally substituted with one or two substituents selected from the group consisting of optionally substituted C₁₋₆alkyl, aryl, heteroaryl, N(R³)₂, OR³, C(O)N(R³)₂, CO₂R³, CN, and halo, wherein R³ is as previously defined.

Other preferred compounds of structural formula (I) are those wherein W is selected from the group consisting of pyridazinyl, pyrimidinyl, pyrazinyl, and triazinyl, optionally substituted with one or two substituents selected from the group consisting of C₁₋₆alkyl, aryl, heteroaryl, N(R³)₂, C(O)N(R³)₂, CO₂R³, OR³, and halo.

In some preferred embodiments, W is selected from the group consisting of

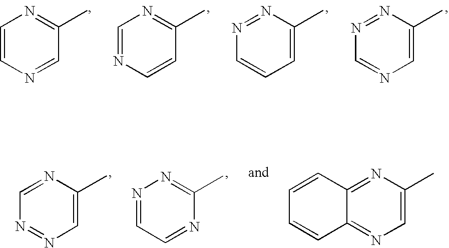

optionally substituted with one to four substituents selected from the group consisting of C₁₋₆alkyl, C₂₋₆ alkynyl, aryl, heteroaryl, CN, CO₂R³, N(R³)₂, OR³, and halo.

In more preferred embodiments, W is

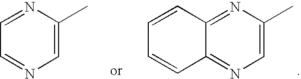

In a most preferred embodiment, W is pyrazinyl and X¹ and X² each are N(H).

In yet another preferred embodiment, the heteroaryl group substituent on W and the heteroaryl group of R⁶, independently, are selected from the group consisting of

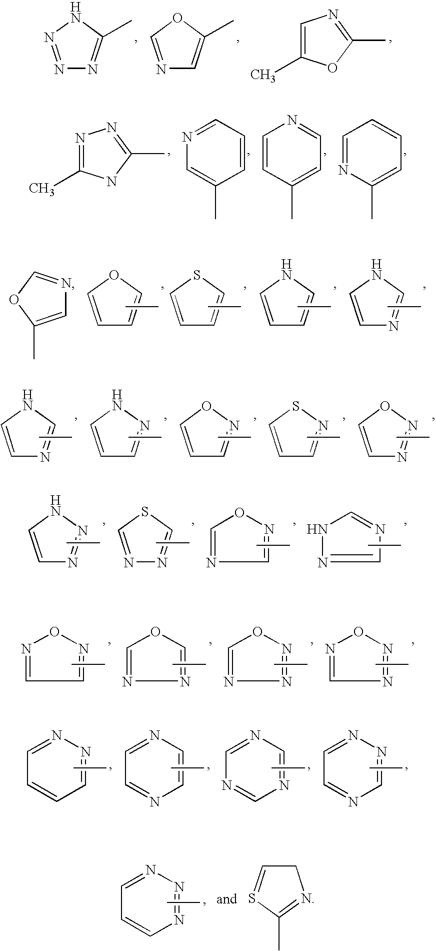

As used herein, the term “alkyl” includes straight chained and branched hydrocarbon groups containing the indicated number of carbon atoms, typically methyl, ethyl, and straight chain and branched propyl and butyl groups. Unless otherwise indicated, the hydrocarbon group can contain up to 20 carbon atoms. The term “alkyl”, includes “bridged alkyl,” i.e., a C₆-C₁₆ bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, cycloalkyl, heterocycloalkyl, amino (N(R³)₂), and sulfonyl (SO₂R³), wherein R³ is as previously defined.

The term “cycloalkyl” is defined as a cyclic C₃₋₈hydrocarbon group, e.g., cyclopropyl, cyclobutyl, cyclohexyl, or cyclopentyl. “Heterocycloalkyl” is defined similarly as cycloalkyl, except the ring contains one to three heteroatoms independently selected from the group consisting of oxygen, nitrogen, and sulfur. Cycloalkyl and heterocycloalkyl groups can be saturated or partially unsaturated ring systems optionally substituted with, for example, one to three groups, independently selected from the group consisting of C₁₋₄alkyl, C₁₋₃alkyleneOH, C(O)NH₂, NH₂, oxo (═O), aryl, trifluoroethanoyl, and OH. Heterocycloalkyl groups optionally can be further N-substituted with C₁₋₆alkyl, hydroxyC₁₋₆alkyl, C₁₋₃alkylenearyl, or C₁₋₃alkyleneheteroaryl.

The term “alkenyl” is defined identically as “alkyl,” except the group contains a carbon-carbon double bond.

The term “alkynyl” is defined identically as “alkyl,” except the group contains a carbon-carbon triple bond.

The term “alkylene” refers to an alkyl group having a substituent. For example, the term “C₁₋₆alkyleneC(O)OR” refers to an alkyl group containing one to six carbon atoms substituted with a —C(O)OR group. The alkylene group is optionally substituted with one or more substituent previously listed as an optional alkyl substituent.

The term “halo” or “halogen” is defined herein as fluorine, bromine, chlorine, and iodine.

The term “aryl,” alone or in combination, is defined herein as a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, C₁₋₆alkyl, C₂₋₆alkenyl, OCF₃, NO₂, CN, NC, N(R³)₂, OR³, CO₂R³, C(O)N—(R³), C(O)R³, N(R¹)COR³, N(R¹)C(O)OR³, N(R)C(O)OR³, N(R¹)C(O)C₁₋₃alkyleneC(O)R³, N(R¹)C(O)C₁₋₃alkyleneC(O)OR³, N(R¹)C(O)C₁₋₃alkyleneOR³, N(R¹)C(O)C₁₋₃alkyleneNHC(O)OR³, N(R¹)C(O)C₁₋₃alkyleneSO₂NR³, C₁₋₃alkyleneOR¹, and SR³, wherein R¹ and R³ are as previously defined. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like. The terms “arylC₁₋₃alkyl” and “heteroarylC₁₋₃alkyl” are defined as an aryl or heteroaryl group having a C₁₋₃alkyl substituent.

The term “heteroaryl”, is defined herein as a monocyclic or bicyclic ring system containing one or two aromatic rings and containing at least one nitrogen, oxygen, or sulfur atom in an aromatic ring. Unless otherwise indicated, a heteroaryl group can be unsub-stituted or substituted with one or more, and in particular one to four, substituents selected from, for example, C₁₋₆alkyl, aryl, heteroaryl, CF₃, CN, C(O)N(R³)₂, CO₂R², N(R³)₂, OR³, and halo, wherein R³ is as previously defined. Examples of heteroaryl groups include, but are not limited to, thienyl, furyl, pyridyl, oxazolyl, quinolyl, isoquinolyl, indolyl, triazinyl, triazolyl, isothiazolyl, isoxazolyl, imidizolyl, benzothiazolyl, pyrazinyl, pyrimidinyl, thiazolyl, and thiadiazolyl.

The term “hydro” is defined as —H.

The term “hydroxy” is defined as —OH.

The term “nitro” is defined as —NO₂.

The term “cyano” is defined as —CN.

The term “isocyano” is defined as —NC.

The term “trifluoromethoxy” is defined as —OCF₃.

The term “azido” is defined as —N₃.

The term “3- to 8-membered ring” as used herein refers to carbocyclic and heterocyclic aliphatic or aromatic groups, including, but not limited to, morpholinyl, piperidinyl, phenyl, thiophenyl, furyl, pyrrolyl, imidazolyl, pyrimidinyl, and pyridinyl, optionally substituted with one or more, and in particular one to three, groups exemplified above for aryl groups.

The carbon atom content of hydrocarbon-containing moieties is indicated by a subscript designating the minimum and maximum number of carbon atoms in the moiety, e.g., “C₁₋₆alkyl”, refers to an alkyl group having one to six carbon atoms, inclusive.

In the structures herein, for a bond lacking a substituent, the substituent is methyl., for example,

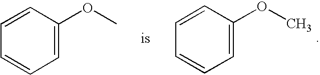

When no substituent is indicated as attached to a carbon atom on a ring, it is understood that the carbon atom contains the appropriate number of hydrogen atoms. In addition, when no substituent is indicated as attached to a carbonyl group or a nitrogen atom, for example, the substituent is understood to be hydrogen, e.g.,

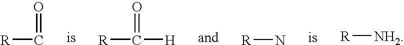

The abbreviation “Me” is methyl. The abbreviation CO and C(O) is carbonyl (C═O).

The notation N(R^{x}) (wherein x represents an alpha or numeric character, such as for example R^{a}, R^{b}, R³, R⁴, and the like) is used to denote two R^{x} groups attached to a common nitrogen atom. When used in such notation, the R^{x} group can be the same or different, and are selected from the group as defined by the R^{x} group.

“Chk1 inhibitor” means any compound, known or after-discovered whether naturally occurring or synthetic, that is capable of at least partially abrogating cell cycle checkpoint activity of the Chk1 protein. Abrogation of cell cycle checkpoint is achieved when the cellular checkpoint mechanisms) is (are) overcome sufficiently to allow the cell to pass from the cell cycle phase in which it is halted to the next phase in the cell cycle or to allow the cell to pass directly to cell death. Abrogation of the cell cycle checkpoint permits cells to carry damage or imperfections to subsequent cell cycle phases, thereby inducing or promoting cell death. Cell death can occur by any associated mechanism, including apoptosis and mitotic catastrophe.

“Chk1 activator” means any known or after-discovered agent having the ability to activate Chk1 kinase activity in DNA repair and homeostasis at cell cycle checkpoints, and thus induce at least partial cell cycle arrest. Chk1 activators include agents capable of arresting the cell cycle at any phase of the cell cycle, which phase may be referred to herein as the “target phase” for that activator. Target phases include any of the cell cycle phases except mitosis, i.e., the G1 phase, S phase, and G2 phase. Chk1 activators useful in the invention include DNA damaging agents, such as chemotherapeutic agents and/or radiation (or “radiotherapeutic agents”), such as ionizing or ultraviolet radiation. Radiation Chk1 activators include, but are not limited to, gamma radiation, X-ray radiation, ultraviolet light, visible light, infrared radiation, microwave radiation, and mixtures thereof.

“Inhibiting aberrant cell proliferation” means to retard or eliminate the rate at which aberrantly proliferating cells proliferate. This inhibition can result either from a decreased rate of replication, an increased rate of cell death, or both. Cell death can occur by any mechanism, including apoptosis and mitotic catastrophe.

“Preventing aberrant cell proliferation” means inhibiting aberrant cell proliferation prior to occurrence, or inhibiting the recurrence thereof.

“In vivo” means within a living subject, as within an animal or human. In this context, agents can be used therapeutically in a subject to retard or eliminate the proliferation of aberrantly replicating cells.

The agents also can be used as a prophylactic to prevent the occurrence or recurrence of aberrant cell proliferation or the manifestation of symptoms associated therewith.

“Ex vivo” means outside a living subject. Examples of ex vivo cell populations include in vitro cell cultures and biological samples such as fluid or tissue samples from humans or animals. Such samples can be obtained by methods well known in the art. Exemplary biological fluid samples include blood, cerebrospinal fluid, urine, saliva. Exemplary tissue samples include tumors and biopsies thereof. In this context, the present compounds can be in numerous applications, both therapeutic and experimental.

The term “radiosensitizer,” as used herein, is defined as a compound, administered to a human or other animal in a therapeutically effective amount to increase the sensitivity of cells to electromagnetic radiation and/or to promote the treatment of diseases treatable with electromagnetic radiation.

The terms “electromagnetic radiation” and “radiation” as used herein include, but are not limited to, radiation having the wavelength of 10-20 to 100 nanometers.

The present invention includes all possible stereoisomers and geometric isomers of the compounds of structural formula (I). The present invention includes not only racemic compounds, but optically active isomers as well. When a compound of structural formula (I) is desired as a single enantiomer, it can be obtained either by resolution of the final product or by stereospecific synthesis from either isomerically pure starting material or use of a chiral auxiliary reagent, for example, see Ma et al., Tetrahedron: Asymmetry, 8(6), pages 883-888 (1997). Resolution of the final product, an intermediate, or a starting material can be achieved by any suitable-method known in the art. Additionally, in situations where tautomers of the compounds of structural formula (I) are possible, the present invention is intended to include all tautomeric forms of the compounds. As demonstrated hereafter, specific stereoisomers can exhibit an exceptional ability to inhibit Chk1 in combination with chemotherapeutic or radiotherapeutic treatments.

Prodrugs of compounds of structural formula (I) also can be used as the compound in a method of the present invention. It is well established that a prodrug approach, wherein a compound is derivatized into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); Silverman, “The Organic Chemistry of Drug Design and Drug Action,” Academic Press, San Diego, chapter 8, (1992); Hillgren et al., Med. Res. Rev., 15, 83 (1995)).

Compounds of the present invention can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as prodrugs.

As used herein, the term “pharmaceutically acceptable salts” refers compounds of structural formula (I) that contain an acidic moiety and form salts having suitable cations. Suitable pharmaceutically acceptable cations include alkali metal (e.g., sodium or potassium) and alkaline earth metal (e.g., calcium or magnesium) cations. In addition, the pharmaceutically acceptable salts of compounds of structural formula (I) that contain a basic center are acid addition salts formed with pharmaceutically acceptable acids. Nonlimiting examples include the hydrochloride, hydrobromide, sulfate, bisulfate, phosphate, hydrogen phosphate, acetate, benzoate, succinate, fumarate, maleate, lactate, citrate, tartrate, gluconate, methanesulfonate, benzene sulphonate, and p-toluenesulphonate salts. In light of the foregoing, any reference to compounds of the present invention appearing herein is intended to include compounds of structural formula (I) as well as pharmaceutically acceptable salts or solvates thereof.

The compounds of the present invention can be therapeutically administered as the neat chemical, but it may be useful to administer compounds of structural formula (I) as a pharmaceutical composition or formulation. Thus, the present invention provides a pharmaceutical composition comprising a compound of the formula (I) together with a pharmaceutically acceptable diluent or carrier therefor. Also provided is a process of preparing a pharmaceutical composition comprising admixing a compound of formula (I) with a pharmaceutically acceptable diluent or carrier therefor.

Accordingly, the present invention further provides pharmaceutical formulations comprising a compound of structural formula (I), or a pharmaceutically acceptable salt, prodrug, or solvate thereof, together with one or more pharmaceutically acceptable carriers and, optionally, other therapeutic and/or prophylactic ingredients. The carriers are “acceptable” in the sense of being compatible with the other ingredients of the formulation and not deleterious to the recipient thereof. Such carriers may be found for example, in Remington's Pharmaceutical Sciences, 179 Ed., Mack Publishing Co., Easton, Pa. (1985).

Inhibition of the checkpoint kinase typically is measured using a dose-response assay in which a sensitive assay system is contacted with a compound of interest over a range of concentrations, including concentrations at which no or minimal effect is observed, through higher concentrations at which partial effect is observed, to saturating concentrations at which a maximum effect is observed. Theoretically, such assays of the dose-response effect of inhibitor compounds can be described as a sigmoidal curve expressing a degree of inhibition as a function of concentration. The curve also theoretically passes through a point at which the concentration is sufficient to reduce activity of the checkpoint enzyme to a level that is 50% that of the difference between minimal and maximal enzyme activity in the assay. This concentration is defined as the “Inhibitory Concentration (50%)” or “IC₅₀” value. Determination of IC₅₀ values may be made using conventional biochemical (acellular) assay techniques or cell-based assay techniques.

Comparisons of the efficacy of inhibitors often are provided with reference to comparative IC₅₀ values, wherein a higher IC₅₀ indicates that the test compound is less potent, and a lower IC₅₀ indicates that the compound is more potent, than a reference compound. Compounds of the present invention demonstrate an IC₅₀ value of less than 5 μM, and down to 0.1 nM, when measured using the dose-response assay. Preferred compounds demonstrate an ICs. value of 500 nM or less. More preferred compounds of the present invention demonstrate an IC₅₀ value of less than 250 nM or less, 100 nM or less, 50 nM or less, 20 nM or less.

Preferred Chk1 inhibitors of the invention are selective, i.e., demonstrate at least a 20-fold selectivity in inhibiting Chk1 over the following protein kinases: protein kinase A, protein kinase C, cdc2, and pp60v-src. More preferred Chk1 inhibitors of the present invention preferably exhibit at least 75-fold selectivity in inhibiting Chk1 over the following protein kinases: protein kinase A, protein kinase C, cdc2, and pp60v-src. Most preferred Chk1 inhibitors of the present invention demonstrate at least 100-fold selectivity against protein kinase A, protein kinase C, cdc2, pp60v-src, protein kinase B/Akt-1, p38MapK, ERK1, p70S6K, cdc2, cdk2, Chk2, and the ab1 tyrosine kinase. “Fold selectivity” is defined as the IC₅₀ of the Chk1 inhibitor for the comparison kinase divided by the IC₅₀ of the Chk1 inhibitor for Chk1.

Compounds and pharmaceutical compositions suitable for use in the present invention include those wherein the active ingredient is administered in an effective amount to achieve its intended purpose. More specifically, a “therapeutically effective amount” means an amount sufficient to treat an individual suffering an indication, or to alleviate the existing symptoms of the indication. Determination of a therapeutically effective amount is well within the capability of those skilled in the art, especially in light of the detailed disclosure provided herein.

In addition to the Chk1 inhibitor, pharmaceutical compositions of the invention can be formulated to include cytokines, lymphokines, growth factors, other hematopoietic factors, or mixtures thereof, to reduce adverse side effects that can arise from, or be associated with, administration of the pharmaceutical composition alone. Cytokines, lymphokines, growth factors, or other hematopoietic factors particularly useful in pharmaceutical compositions of the invention include, but are not limited to, M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF, G-CSF, Meg-CSF, GM-CSF, thrombopoietin, stem cell factor, erythropoietin, angiopoietins, including Ang-1, Ang-2, Ang-4, Ang-Y, and/or the human angiopoietin-like polypeptide, vascular endothelial growth factor (VEGF), angiogenin, bone morphogenic protein-1 (BMP-1), BMP-2, BMP-3, BMP-4, BMP-5, BMP-6, BMP-7, BMP-8, BMP-9, BMP-10, BMP-11, BMP-12, BMP-13, BMP-14, BMP-15, BMP receptor IA, BMP receptor IB, brain derived neurotrophic factor, ciliary neutrophic factor, ciliary neutrophic factor receptor cytokine-induced neutrophil chemotactic factor 1, cytokine-induced neutrophil chemotactic factor 2, cytokine-induced neutrophil chemotactic factor 2, endothelial cell growth factor, endothelin 1, epidermal growth factor, epithelial-derived neutrophil attractant, fibroblast growth factor (FGF) 4, FGF 5, FGF 6, FGF 7, FGF 8, FGF 8b, FGF 8c, FGF 9, FGF 10, FGF acidic, FGF basic, glial cell line-derived neutrophic factor receptor 1, glial cell line-derived neutrophic factor receptor 2, growth related protein, growth related protein, growth related protein, growth related protein, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor, platelet derived growth factor receptor, pre-B cell growth stimulating factor, stem cell factor, stem cell factor receptor, transforming growth factor (TGF), TGF, TGF 1, TGF 1.2, TGF 2, TGF 3, TGF 5, latent TGF 1, TGF, binding protein I, TGF binding protein II, TGF binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

The compounds of structural formula (I) also can be conjugated or linked to auxiliary moieties that promote a beneficial property of the compound in a method of therapeutic use. Such conjugates can enhance delivery of the compounds to a particular anatomical site or region of interest (e.g., a tumor), enable sustained therapeutic concentrations of the compounds in target cells, alter pharmacokinetic and pharmacodynamic properties of the compounds, and/or improve the therapeutic index or safety profile of the compounds. Suitable auxiliary moieties include, for example, amino acids, oligopeptides, or polypeptides, e.g., antibodies such as monoclonal antibodies and other engineered antibodies; and natural or synthetic ligands to receptors in target cells or tissues. Other suitable auxiliaries include fatty acid or lipid moieties that promote biodistribution and/or uptake of the compound by target cells (see, e.g., Bradley et al., Clin. Cancer Res. (2001) 7:3229).

Formulations of the present invention can be administered in a standard manner for the treatment of the indicated diseases, such as by oral, parenteral, transmucosal (e.g., sublingual or via buccal administration), topical, transdermal, rectal, or inhalation (e.g., nasal or deep lung inhalation) administration. Parenteral administration includes, but is not limited to intravenous, intraarterial, intraperitoneal, subcutaneous, intramuscular, intrathecal, and intraarticular modes of administration. Parenteral administration also can be accomplished using a high pressure technique, like POWDERJECT™.

For oral administration and buccal administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch, or polyvinylpyrrolidone), fillers (for example, lactose, sugar, microcrystalline cellulose, maize-starch, calcium phosphate, or sorbitol), lubricants (for example, magnesium stearate, stearic acid, talc, polyethylene glycol or silica), disintegrants (for example, potato starch or sodium starch glycolate), or wetting agents (for example, sodium lauryl sulfate). The tablets can be coated according to methods well known in the art.

Alternatively, compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, for example suspending agents, such as sorbitol syrup, methyl cellulose, glucose/sugar syrup, gelatin, hydroxyethylcellulose, hydroxypropylmethylcellulose, carboxymethylcellulose, aluminum stearate gel, and hydrogenated edible fats; emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid.

Such preparations also can be formulated as suppositories, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions of the present invention can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition of the present invention also can be formulated as a depot preparation. Such long acting formulations can be administered by implantation (for example, subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

For veterinary use, a compound of formula (I), or a pharmaceutically acceptable salt, prodrug, or solvent thereof, is administered as a suitably acceptable formulation in accordance with normal veterinary practice. The veterinarian can readily determine the dosing regimen and route of administration that is most appropriate for a particular animal. Animals treatable by the present compounds and methods include, but are not limited to, pets, livestock, show animals, and zoo specimens.

### Synthetic Methods

Compounds of the present invention can be prepared by the following synthetic schemes. Starting materials can be obtained from commercial sources or prepared by well-established literature methods known to those of ordinary skill in the art. The groups X, R¹, R³, R⁴, R⁵, R⁶, R⁷, R⁸, R⁹, R¹⁰, and R¹¹ are defined above unless otherwise noted below. R² is as defined above, and also includes CF₃ and heteroaryl in the following synthetic schemes.

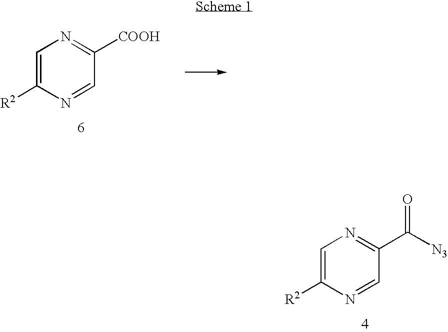

As illustrated in Scheme 1, compounds of formula 4 can be prepared from compounds of formula 6 by treatment with a base, such as DIEA, and diphenyl phosphoryl azide. A typical solvent for this reaction is THF, and the reaction is performed behind a blast shield at room temperature over a one- to twelve-hour period.

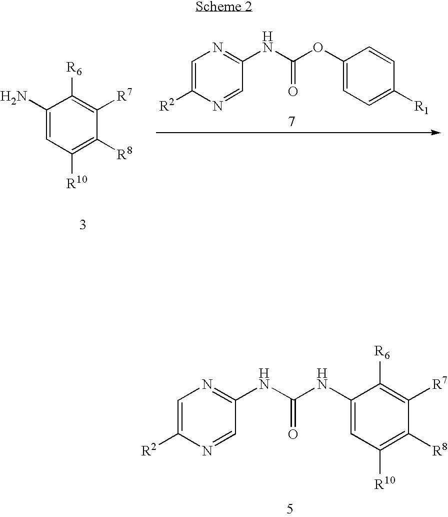

Scheme 2 shows an alternative synthesis of compounds of formula 5. Compounds of formula 3 are treated with compounds of formula 7, which is prepared according to Scheme 3. A useful, nonlimiting solvent is DMF, and the reaction temperature is maintained between room temperature and 60° C. for about one to twelve hours.

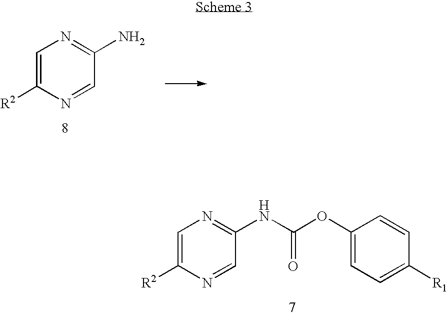

As demonstrated in Scheme 3, compounds of formula 7 can be prepared from compounds of formula 8 by treatment with an aryl chloroformate, such as phenyl chloroformate or p-nitrophenyl chloroformate, in the presence of a base, such as pyridine. Nonlimiting solvents used in this reaction include CH₂Cl₂ or pyridine, at temperatures from 0° C. to room temperature.

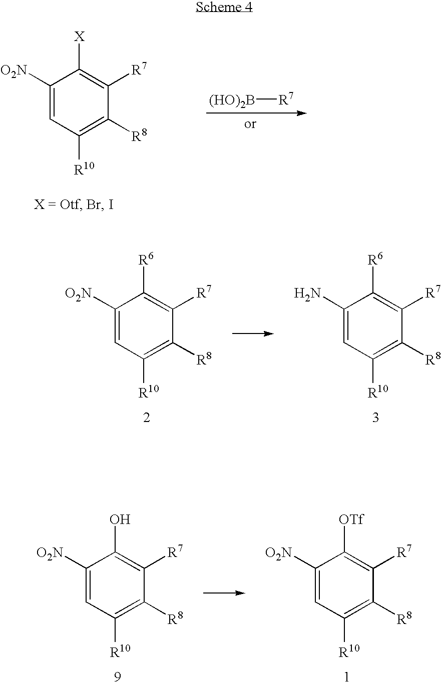

Scheme 4 shows an approach to compounds of formula 3. Compounds of formula 1 are converted to compounds of formula 2 by treatment with aryl boronic acids and a source of palladium(0) (for example, palladium tetrakis triphenylphosphine) in the presence of a basic aqueous solution, such as sodium bicarbonate, potassium carbonate, or potassium phosphate. Nonlimiting examples of solvents used in this reaction include THF, dioxane, or ethylene glycol dimethyl ether. The reaction typically is performed at temperatures between 0° C. and 90° C. for about 1 to 12 hours. Compounds of formula 2 are converted to compounds of formula 3 in the presence of palladium on carbon, platinum on carbon, or zinc, for example. Examples of solvents used in this reaction include, but are not limited to, methanol, ethanol, or acetic acid. Alternatively, compounds of formula 1 can be used to arylate terminal alkynes, 11, using a catalyst, such as dichloro palladium bis triphenyl phosphine or any other source of palladium(0). Reactions typically are conducted at temperatures varying from room temperature to 90° C., in the presence of a base, such as trimethylamine.

Furthermore, compounds of formula 1, where X is a triflate, i.e., tf, can be obtained from compounds of formula 9. Typical reagents include triflic anhydride or N-phenyl triflimide. The reaction typically is performed at temperatures between −10° C. and room temperature. A nonlimiting example of a solvent is dichloromethane. Nonlimiting examples of bases are triethylamine or di-isopropyl ethyl amine.

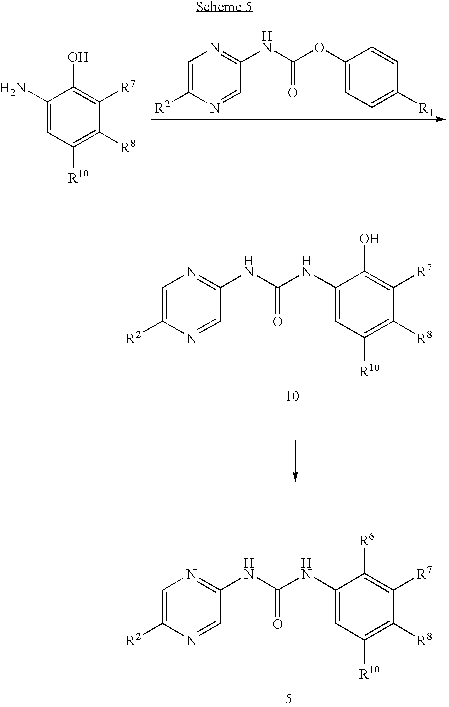

Scheme 5 illustrates an alternative synthesis for compounds of formula 5. Compounds of formula 3 can be converted to compounds of formula 10 following procedures described in Scheme 2. Compounds of formula 10 then can be converted to compounds of formula 5 following procedures described in Scheme 4.

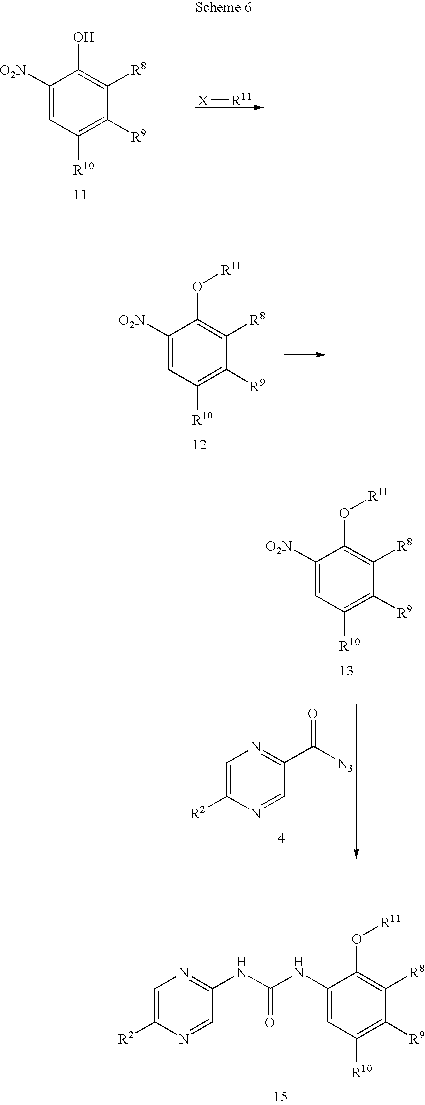

As shown in Scheme 6, compounds of formula 11 can be converted to compounds of formula 12 by treatment with a base, such as potassium carbonate, triethylamine, or sodium hydride, followed by the addition of R¹¹X, wherein X is a halide, mesylate, or tosylate. Examples of solvents used in this reaction include DMF, THF, CH₂Cl₂, and mixtures thereof. The reaction is conducted at temperatures between 0° C. and 100° C. for about 15 minutes to 12 hours.

Alternatively, compounds of formula 11 can be admixed with a compound of formula R¹¹X, wherein X is hydroxyl, and the resulting mixture is treated with triphenylphosphine and diisopropylazodicarboxylate in a solvent, such as THF, to provide compounds of formula 12.

Compounds of formula 12 can be treated with hydrogen gas in the presence of a catalyst such as platinum oxide, palladium on carbon, or Raney nickel, or treated with an acid source, such as saturated aqueous ammonium chloride or aqueous hydrogen chloride in the presence of zinc metal, to provide compounds of formula 13. Examples of solvents used in this reaction include methanol, ethanol, ethyl acetate, or mixtures thereof. The reaction generally is conducted at room temperature or below for periods of one to twelve hours.

Compounds of formula 15 can be prepared by combining compounds of formula 13 with compounds of formula 4 (prepared as described in Scheme 1). Examples of solvents used in this reaction include toluene, benzene, and xylene. The reaction is performed at temperatures of 60° C. to 100° C. for five to twelve hours.

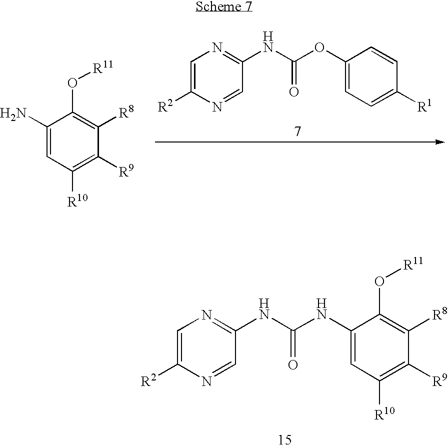

Scheme 7 shows an alternative synthesis of compounds of formula 15. Compounds of formula 3 are treated with compounds of formula 7, which is prepared according to Scheme 3. One solvent that can be used is DMF, and the reaction temperature is maintained between room temperature and 60° C. over a one- to twelve-hour time period.

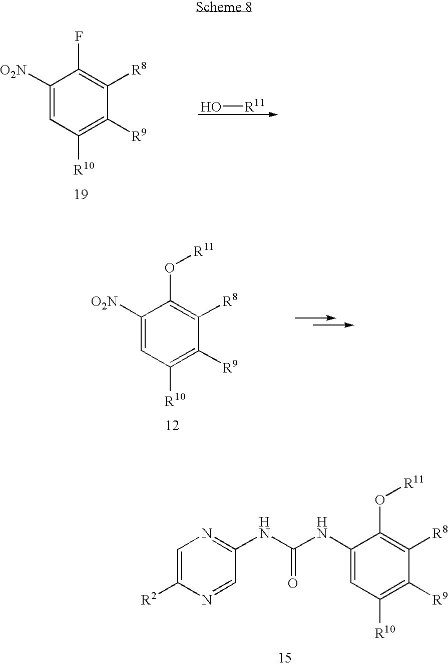

Scheme 8 shows an alternative approach to compounds of formula 15. Compounds of formula 19 are converted to compounds of formula 12 by treatment with an alcohol in the presence of a base, such as sodium hydride, potassium bis(trimethylsilyl)amide, or n-butyllithium. Examples of solvents used in this reaction include THF or diethyl ether. The reaction typically is performed at temperatures between −15° C. and room temperature for about 1 to 6 hours. Compounds of formula 12 are converted to compounds of formula 15 following procedures described in Scheme 6.

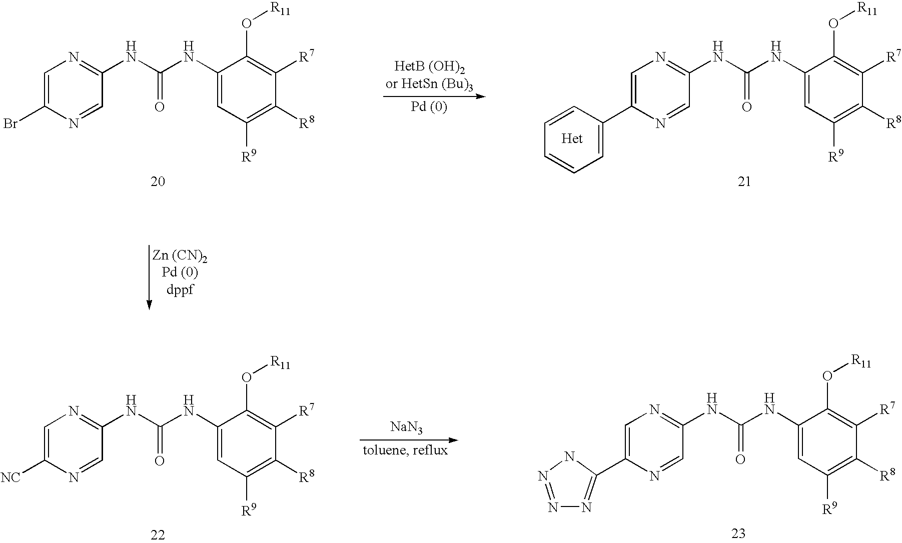

Scheme 9 shows an alternative synthesis of compounds 21 and 23. Compound 20 can be synthesized using procedures described in Scheme 5 or Scheme 6, wherein R₂=Br. Compound 20 can be converted to Compound 21 by treatment with heteroaryl boronic acids (HetB(OH)₂) and a source of palladium(0) (for example, palladium tetrakis triphenylphosphine) in the presence of a basic aqueous solution, such as sodium bicarbonate, potassium carbonate, or potassium phosphate. Nonlimiting examples of solvents used in this reaction include THF, dioxane, or ethylene glycol dimethyl ether. The reaction typically is performed at temperatures between 0° C. and 90° C. for about 1 to 12 hours. Alternatively, an heteroaryl boronic acid can be replaced with a heteroaryl stannate, e.g., (HetSn(Bu)₃).

Compound 21 can also be converted to 22 by treatment, for example, with zinc cyanide and a source of palladium(0) (for example, palladium tetrakis triphenylphosphine), in the presence of a base such as triethylamine or Hunig's base. Nonlimiting examples of solvents used in this reaction are DMF and DME, at 80° C. for 1 to 12 hours. Alternatively, compound 22 can be obtained from 21, using potassium cyanide, in the presence of a source of Pd(0) (such as palladium tetrakis triphenyl phosphine), and a source of copper (such as copper iodide). The reaction is typically performed at temperatures between room temperature and 200° C., for about 30 minutes to 5 hours. Nonlimiting examples of solvents used in this reaction include DMF.

Finally, Compound 22 can be converted to Compound 23, using for example sodium azide, in the presence of a base such as triethylamine. Nonlimiting examples of solvents used in this reaction include DMF or nitrobenzene. The reaction typically is conducted at temperatures between 80° C. and 100° C., for about 1 to 5 hours.

Specific, nonlimiting examples of compounds of structural formula (I) are provided below, the synthesis of which were performed in accordance with the procedures set forth below and in copending U.S. Patent Application Publication No. 2003-0069284 A1, incorporated herein by reference.

Abbreviations used in the syntheses described herein are: hours (h), water (H₂O), magnesium sulfate (MgSO₄), hydrochloric acid (HCl), dimethyl sulfoxide (DMSO), diisopropyl azodicarboxylate (DIAD), methylene chloride (CH₂Cl₂), chloroform (CHCl₃), methanol (MeOH), ammonium hydroxide (NH₄OH), deuterated chloroform (CDCl₃), tetrahydrofuran (THF), N-methylpyrrolidone (NMP), acetic acid (AcOH), sodium hydroxide (NaOH), ethyl acetate (EtOAc), ethanol (EtOH), dimethyl sulfoxide (DMSO), diethyl ether (Et₂O), sodium carbonate (Na₂CO₃), sodium bicarbonate (NaHCO₃), nitric acid (HNO₃), sodium chloride (NaCl), sodium sulfate (Na₂SO₄), dimethylformamide (DMF), 1,8-diazabicyclo[5.4.0]undec-7-ene (DBU), and N,N-diisopropylethylamine (DIEA).

### Intermediate 1:

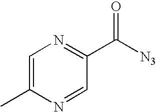

### 5-Methyl-pyrazine-2-carbonyl azide

To a stirred suspension of 5-methyl-pyrazine-2-carboxylic acid (25 g, 181 mmol) in 540 mL THF at room temperature under nitrogen was added DIEA (31.7 mL, 181 mmol) resulting in a brown solution. Diphenyl phosphoryl azide (39.2 mL, 181 mmol) then was added dropwise as a solution in 50 mL THF over 1 hour behind a blast shield. The reaction was allowed to stir overnight. The reaction then was rotary evaporated to a small volume at room temperature and partitioned between Et₂O (1 L) and H₂O (1 L). The H₂O layer was back extracted with 2×250 mL Et₂O, and the combined organics washed 2×μL with saturated sodium bicarbonate. The organics were dried (MgSO₄), filtered, and concentrated to a solid mass, which was triturated with Et₂O to give the product as a yellow solid (15g, 50%). Purer compound could be isolated by taking x g of the crude product in 20×mL of Et₂O, and treating with 1-2×g of decolorizing carbon at room temperature for a few minutes. After filtration and concentration, this material was homogeneous by TLC in EtOAc and pure white. The recovery was typically 65%.

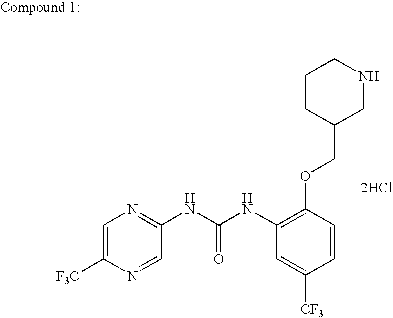

### 1-[2-(Piperidin-3-ylmethoxy)-5-trifluoromethyl-phenyl]-3-(5-trifluoromethyl-pyrazin-2-yl)-urea hydrochloride salt

Step 1: 3-[2-(4-Nitro-phenoxycarbonylamino)-4-trifluoromethyl-phenoxymethyl]-piperidine-1-carboxylic acid tert-butyl ester. To a stirred solution of 3-(2-amino-4-trifluoromethyl-phenoxymethyl)-piperidine-1-carboxylic acid tert-butyl ester (240 mg, 0.64 mmol) in 2 mL CH₂Cl₂ at 0° C. under nitrogen was added pyridine (57 uL, 0.71 mmol) followed by p-nitrophenyl chloroformate (130 mg, 0.64 mmol). After 1 hour at 0° C., the reaction mixture was diluted to 30 mL with CH₂Cl₂, then washed 2×30 mL with 2N HCl, 1×30 mL with water, and 1×30 mL with brine. The organics were dried (MgSO₄), filtered, and concentrated to an off white foam.

Step 2: 3-(4-Trifluoromethyl-2-[3-(5-trifluoromethyl-pyrazin-2-yl)-ureido)-phenoxymethyl]-piperidine-1-carboxylic acid tert-butyl ester. 3-[2-(4-Nitro-phenoxycarbonylamino)-4-trifluoromethyl-phenoxymethyl]-piperidine-1-carboxylic acid tert-butyl ester (217 mg, 0.4 mmol) and 5-trifluoromethyl-pyrazin-2-ylamine (66 mg, 0.4 mmol) (prepared according to the method of U.S. Pat. No. 4,293,552) were mixed as solids in a S mL reaction vial, diluted with 400 uL NMP, capped and stirred as a dark yellow solution that then was immersed in an oil bath at 85° C. and stirred for six hours. The reaction mixture was cooled to room temperature and stirred overnight. NMP then was removed by Kugelrohr distillation at 0.5 mm and 80° C. The brown residue was diluted to 30 mL with CH₂Cl₂ then washed 3×30 mL with 1M Na₂CO₃ to remove p-nitrophenol. The organics were dried (MgSO₄), filtered, and concentrated to a crude solid that was triturated with EtOAc and filtered to give the desired product as a white solid.

Step 3: To a stirred solution of 3-[2-(4-nitro-phenoxycarbonylamino)-4-trifluoromethyl-phenoxymethyl]-piperidine-1-carboxylic acid tert-butyl ester (60 mg, 0.11 mmol) in 2 mL dioxane at room temperature in a capped flask was added 2N HCl in dioxane (2 mL) and the reaction mixture was stirred overnight. The resulting suspension was concentrated by rotary evaporation and high vacuum to give a yellow solid corresponding to the final product (57 mg, 99%). ¹H-NMR (400 MHz, d₆-DMSO) δ: 11.18 (br s, 1H), 9.91 (s, 1H), 9.13 (s, 1H), 8.79 (s, 1H), 8.62 (br s, 1H), 8.57 (s, 1H), 7.43 (d, 1H), 7.25 (d, 1H), 4.18 (m, 2H), 3.44 (m, 1H), 3.23 (m, 1H), 2.86 (m, 2H), 2.36 (m, 1H), 1.96 (m, 1H), 1.83 (m, 1H), 1.67 (m, 1H), 1.42 (m, 1H). LRMS (apci, positive) m/e 464.3 (M+1).

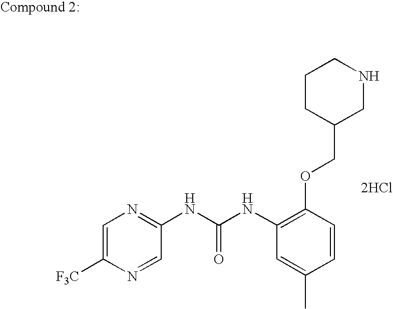

### 1-[5-Methyl-2-(piperidin-3-ylmethoxy)-phenyl]-3-(5-trifluoromethyl-pyrazin-2-yl)-urea hydrochloride salt

Step 1: 3-{4-Methyl-2-[3-(5-trifluoromethyl-pyrazin-2-yl)-ureido]-phenoxymethyl}-piperidine-1-carboxylic acid tert-butyl ester. Prepared from 3-[4-methyl-2-(4-nitro-phenoxycarbonylamino)-phenoxymethyl]-piperidine-1-carboxylic acid tert-butyl ester (WO 02/070494) according to the procedure of Compound I, Step 2. The product was isolated as a white solid.

Step 2: Prepared from 3-{4-methyl-2-[3-(5-trifluoromethyl-pyrazin-2-yl)-ureido]-phenoxymethyl}piperidine-1-carboxylic acid tert-butyl ester according to the procedure of Compound I, Step 3. The final product was isolated as a yellow solid (30 mg, 99%). ¹H-NMR (400 MHz, d₆-DMSO) δ: 10.86 (s, 1H), 9.61 (s, 1H), 9.05 (s, 1H), 8.77 (s, 1H), 8.58 (br s, 1H), 7.98 (s, 1H), 6.98 (d, 1H), 6.83 (d, 1H), 3.99 (m, 2H), 3.40 (m, 1H), 3.23 (m, 1H), 2.81 (m, 2H), 2.23 (m, 1H), 2.22 (s, 3H), 1.91 (m, 1H), 1.80 (m, 1H), 1.62 (m, 1H), 1.39 (m, 1H). LRMS (apci, positive) m/e 410.4 (M+1).

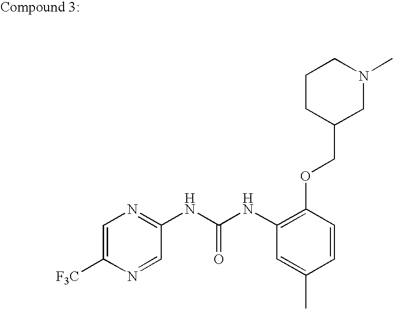

### 1-[5-Methyl-2-(1-methyl-piperidin-3-ylmethoxy)-phenyl]-3-(5-trifluoromethyl-pyrazin-2-yl)-urea

Prepared according to the procedure of Compound I Step 2 from [5-methyl-2-(1-methyl-piperidin-3-ylmethoxy)-phenyl]-carbamic acid 4-nitro-phenyl ester (WO 02/070494) as a white solid. ¹H-NMR (400 MHz, CDCl₃) δ: 11.01 (br 5, 1H), 8.87 (br s, 1H), 8.82 (s, 1H), 8.44 (s, 1H), 8.17 (s, 1H); 6.84 (d, 1H), 6.76 (d, 1H), 3.84 (m, 2H), 3.16 (m, 1H), 2.81 (m, 1H), 2.37 (s, 3H), 2.29 (m, 1H), 2.20 (s, 3H), 1.99 (m, 1H), 1.87-1.62 (m, 4H), 1.11 (m, 1H). LRMS (apci, positive) m/e 424.3 (M+1).

Additional nonlimiting compounds of the present invention are illustrated below.

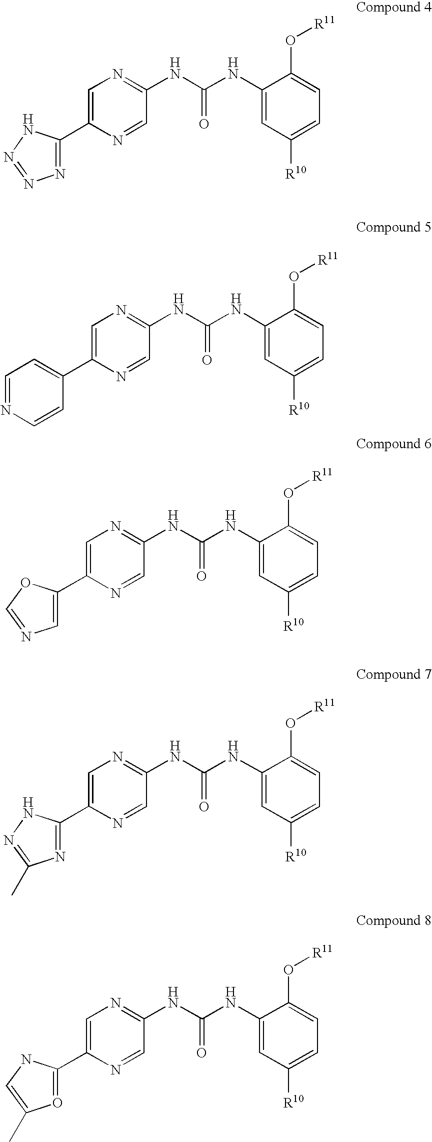

### Therapeutic Methods

Compounds of the present invention can be used to potentiate the therapeutic effects of radiation and/or a chemotherapeutic agent used in the treatment of cancers and other cell proliferation indications involving eukaryotic cells, including those in humans and other animals. For example, compounds of the invention can be used to enhance treatment of tumors that are customarily treated with an antimetabolite, e.g., methotrexate or 5-fluorouracil (5-FU). In general, the present compounds inhibit aberrantly proliferating cells, both cancerous and noncancerous.

Use of compounds of the present invention can result in partial or complete regression of aberrantly proliferating cells, i.e., the partial or complete disappearance of such cells from the cell population. Thus, for example, when the population of aberrantly proliferating cells are tumor cells, the method of the invention can be used to slow the rate of tumor growth, decrease the size or number of tumors, or to induce partial or complete tumor regression.

In all embodiments, the invention can be used in vivo or ex vivo where no aberrant cell proliferation has been identified or where no aberrant cell proliferation is ongoing, but where aberrant cell proliferation is suspected or expected, respectively. Moreover, the invention also can be used wherever aberrant cell proliferation has been previously treated to prevent or inhibit recurrence of the same. In these and related embodiments, the “cell population comprising aberrantly proliferating cells” refers to any cell population where no aberrant cell proliferation has been identified or is ongoing, but where aberrant cell proliferation is suspected or expected, respectively, and/or any cell population previously treated for aberrant cell proliferation to prevent or inhibit recurrence of the same.

One method of the present invention comprises administration of a therapeutically effective amount of a present Chk1 inhibitor compound in combination with a chemotherapeutic agent that can effect single- or double-strand DNA breaks or that can block DNA replication or cell proliferation. Alternatively, a method of the present invention comprises administration of a therapeutically effective amount of at least one of the present Chk1 inhibitor compounds in combination with therapies that include use of an antibody, e.g., herceptin, that has activity in inhibiting the proliferation of cancer cells. Accordingly, cancers, for example, colorectal cancers, head and neck cancers, pancreatic cancers, breast cancers, gastric cancers, bladder cancers, vulvar cancers, leukemias, lymphomas, melanomas, renal cell carcinomas, ovarian cancers, brain tumors, osteosarcomas, and lung carcinomas, are susceptible to enhanced treatment by administration of a present Chk1 inhibitor in combination with a chemotherapeutic agent or an antibody.

Cancers include tumors or neoplasms which are growths of tissue cells wherein multiplication of cells is uncontrolled and progressive. Some such growths are benign, but others are termed “malignant,” and can lead to death of the organism. Malignant neoplasms, or “cancers,” are distinguished from benign growths in that, in addition to exhibiting aggressive cellular proliferation, can invade surrounding tissues and metastasize. Moreover, malignant neoplasms are characterized by showing a greater loss of differentiation (greater “dedifferentiation”) and organization relative to one another and surrounding tissues. This property is called “anaplasia.”

Cancers treatable by the present invention also include solid tumors, i.e., carcinomas and sarcomas. Carcinomas include malignant neoplasms derived from epithelial cells which infiltrate (i.e., invade) surrounding tissues and give rise to metastases. Adenocarcinomas are carcinomas derived from glandular tissue, or from tissues that form recognizable glandular structures. Another broad category of cancers includes sarcomas, which are tumors whose cells are embedded in a fibrillar or homogeneous substance, like embryonic connective tissue. The present invention also enables treatment of cancers of the myeloid or lymphoid systems, including leukemias, lymphomas, and other cancers that typically are not present as a tumor mass, but are distributed in the vascular or lymphoreticular systems.

Chk1 activity is associated with various forms of cancer in, for example, adult and pediatric oncology, growth of solid tumors/malignancies, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma), gastrointestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, and squamous cell cancer), thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. Accordingly, administration of a present Chk1 inhibitor is expected to enhance treatment regimens.

Compounds of the present invention also can potentiate the efficacy of drugs in the treatment of inflammatory diseases. Examples of diseases that can benefit from combination therapy with compounds suitable for the method of the present invention are rheumatoid arthritis, psoriasis, vitiligo, Wegener's granulomatosis, and systemic lupus erythematosus (SLE). Treatment of arthritis, Wegener's granulomatosis, and SLE often involves the use of immunosuppressive therapies, such as ionizing radiation, methotrexate, and cyclophosphamide. Such treatments typically induce, either directly or indirectly, DNA damage. Inhibition of Chk1 activity within the offending immune cells render the cells more sensitive to control by these standard treatments. Psoriasis and vitiligo commonly are treated with ultraviolet radiation (UV) in combination with psoralen. The present DNA damaging agents induce the killing effect of UV and psoralen, and increase the therapeutic index of this treatment regimen. In general, compounds useful in methods of the present invention potentiate control of inflammatory disease cells when in combination with currently used immunosuppressive drugs.

In addition to the cancers disclosed above, the present invention also can be used in methods of treating noncancerous proliferating cells. Such conditions include, but are not limited to, atherosclerosis, restenosis, vasculitis, nephritis, retinopathy, renal disease, proliferative skin disorders, psoriasis, keloid scarring, actinic keratosis, Stevens-Johnson Syndrome, rheumatoid arthritis (PA), systemic-onset juvenile chronic arthritis (JCA), osteoporosis, systemic lupus erythmatosis, hyperproliferative diseases of the eye including epithelial down growth, proliferative vitreoretinopathy (PVR), diabetic retropathy, Hemarigio-proliferative diseases, ichthyosis, or papillomas.

One preferred method of administering a Chk1 inhibitor of the present invention is described in Keegan et al., U.S. Provisional application No. 60/503,925, filed Sep. 17, 2003, the entire disclosure of which is incorporated herein by reference herein. Such methods for inhibiting aberrant cell proliferation involve scheduling the administration of a Chk1 activator (e.g., a chemotherapeutic agent) and a Chk1 inhibitor according to the present invention. In this method, at least one Chk1 activator is administered at a dose and for a time sufficient to induce substantial synchronization of cell cycle arrest in proliferating cells. Upon achieving substantial phase synchronization, at least one Chk1 inhibitor is administered to abrogate the cell cycle arrest and induce therapeutic cell death. The method is useful with any Chk1 activator, and finds application in treating or preventing cancerous and noncancerous aberrant cell proliferation.

A population of aberrantly proliferating cells can be contacted with one Chk1 inhibitor or can be contacted with more than one Chk1 inhibitor. If more than one Chk1 inhibitor is used, the Chk1 inhibitors can be coadministered or administered at separate times as determined by the attending physician or laboratory technician.

A population of aberrantly proliferating cells also can be contacted with one Chk1 activator or can be contacted with more than one Chk1 activator. If more than one Chk1 activator is used, the Chk1 activators can be coadministered or administered at separate times as determined by the attending physician or laboratory technician.

The Chk1 inhibitors of the present invention can be employed to study or modify the behavior of cell populations ex vivo. For example, the present compounds can be used ex vivo to determine the optial schedule and/or dosing of administration of a Chk1 inhibitor for a given indication, cell type, patient, and other parameter. Information gleaned from such use can be used for experimental purposes or in the clinic to set protocol for in vitro treatment. Other ex vivo uses for which the invention is suited are apparent to those skilled in the art.

A Chk1 inhibitor compound of the present invention also can radiosensitize a cell. Diseases that are treatble with electromagnetic radiation include neoplastic diseases, benign and malignant tumors, and cancerous cells.

Electromagnetic radiation treatment of other diseases not listed herein also is contemplated by the present invention. Preferred embodiments of the present invention employ the electromagnetic radiation of: gamma radiation (10⁻²⁰ to 10⁻¹³ m), X-ray radiation (10⁻¹² to 10⁻¹⁹ m), ultraviolet light (10 nm to 400 nm), visible light (400 nm to 700 nm), infrared radiation (700 nm to 1.0 mm), and microwave radiation (1 mm to 30 cm).

Many cancer treatment protocols currently employ radiosensitizers activated by electromagnetic radiation, e.g., X-rays. Examples of X-ray-activated radiosensitizers include, but are not limited to, the following: metronidazole, misonidazole, desmethylmisonidazole, pimonidazole, etanidazole, nimorazole, mitomycin C, RSU 1069, SR 4233, E09i RB 6145, nicotinamide, 5-bromodeoxyuridine (BudR), 5-iododeoxyuridine (IUdR), bromodeoxycytidine, fluorodeoxyuridine (FUdR), hydroxyurea, cis-platin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, PHOTOFRIN®, benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Radiosensitizers can be administered in conjunction with a therapeutically effective amount of one or more compounds in addition to the Chk1 inhibitor, such compounds including, but not limited to, compounds that promote the incorporation of radiosensitizers to the target cells, compounds that control the flow of therapeutics, nutrients, and/or oxygen to the target cells, chemotherapeutic agents that act on the tumor with or without additional radiation, or other therapeutically effective compounds for treating cancer or other disease. Examples of additional therapeutic agents that can be used in conjunction with radiosensitizers include, but are not limited to, 5-fluorouracil (5-FU), leucovorin, oxygen, carbogen, red cell transfusions, perfluorocarbons (e.g., FLUOSOL®-DA), 2,3-DPG, BW12C, calcium channel blockers, pentoxifylline, antiangiogenesis compounds, hydralazine, and L-BSO.

Chemotherapeutic agents that can be used include, but are not limited to, alkylating agents, antimetabolites, hormones and antagonists thereof, radioisotopes, antibodies, as well as natural products, and combinations thereof. For example, an inhibitor compound of the present invention can be administered with antibiotics, such as doxorubicin and other anthracycline analogs, nitrogen mustards, such as cyclophosphamide, pyrimidine analogs such as 5-fluorouracil, cis-platin, hydroxyurea, taxol and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Other antineoplastic protocols include the use of an inhibitor compound with another treatment modality, e.g., surgery or radiation, also referred to herein as “adjunct anti-neoplastic modalities.” Additional chemotherapeutic agents useful in the invention include hormones and antagonists thereof, radioisotopes, antibodies, natural products, and combinations thereof. Examples of chemotherapeutic agents useful for the method of the present invention are listed in the following table.

| | | |
|---|---|---|
| Alkylating agents | Nitrogen mustards | mechlorethamine |
| cyclophosphamide | ifosfamide | melphalan |
| chlorambucil | Nitrosoureas | carmustine (BCNU) |
| lomustine (CCNU) | semustine (methyl- | Ethylenimine/Methylmelamine |
| | CCNU) | |
| thriethylenemelamine (TEM) | triethylene | (thiotepa) |
| | thiophosphoramide | |
| hexamethylmelamine | (HMM, altretaroine) | Alkyl sulfonates |
| busulfan | Triazines | dacarbazine (DTIC) |
| Antimetabolites | Folic Acid analogs | methotrexate |
| trimetrexate | Pyrimidine analogs | 5-fluorouracil |
| fluorodeoxyuridine | gemcitabine | cytosine arabinoside |
| (AraC, cytarabine) | 5-azacytidine | 2,2′-difluorodeoxycytidine |
| Purine analogs | 6-mercaptopurine | 6-thioguanine |
| azathioprine | 2′-deoxycoformycin | (pentostatin) |
| erythrohydroxynonyladenine(EHNA) | fludarabine phosphate | 2-chlorodeoxyadenosine |
| (cladribine, 2-CdA) | multitargeted | Type I Topoisomerase |
| | antifolate | Inhibitors |
| camptothecin | topotecan | irinotecan |
| Natural products | Antimitotic drugs | paclitaxel |
| Vinca alkaloids | vinblastine (VLB) | vincristine |
| vinorelbine | Taxotere (docetaxel) | estramustine |
| estramustine phosphate | etoposide | teniposide |
| Epipodophylotoxins | | |
| Antibiotics | actimomycin D | daunomycin (rubidomycin) |
| doxorubicin (adriamycin) | mitoxantroneidarubicin | bleomycinsplicamycin |
| | | (mithramycin) |
| mitomycinC | dactinomycin | Enzymes |
| L-asparaginase | Biological response | interferon-alpha |
| | modifiers | |
| IL-2 | G-CSF | GM-CSF |
| Differentiation Agents | retinoic acid | Radiosensitizers |
| | derivatives | |
| metronidazole | misonidazole | desmethylmisonidazole |
| pimonidazole | etanidazole | nimorazole |
| RSU 1069 | EO9 | RB 6145 |
| SR4233 | nicotinamide | 5-bromodeozyuridine |
| 5-iododeoxyuridine | bromodeoxycytidine | Miscellaneous agents |
| Platinium coordination | cis-platin | carboplatin |
| complexes | | |
| oxaliplatin | Anthracenedione | mitoxantrone |
| Substituted urea | hydroxyurea | Methylhydrazine derivatives |
| N-methylhydrazine (MIH) | procarbazine | Adrenocortical suppressant |
| mitotane (o,p′-DDD) | ainoglutethimide | Cytokines |
| interferon (α, β, γ) | interleukin-2 | Adrenocorticosteroids/ |
| | Hormones and | antagonists |
| | antagonists | |
| prednisone and equivalents | dexamethasone | ainoglutethimide |
| Progestins | hydroxyprogesterone | medroxyprogesterone acetate |
| | caproate | |
| megestrol acetate | Estrogens | diethylstilbestrol |
| ethynyl estradiol/ | Antiestrogen | tamoxifen |
| equivalents | | |
| Androgens | testosterone | fluoxymesterone/equivalents |
| | propionate | |
| Antiandrogens | flutamide | gonadotropin-releasing |
| hormone analogs | leuprolide | Nonsteroidal antiandrogens |
| flutamide | Photosensitizers | hematoporphyrin derivatives |
| Photofrin | benzoporphyrin | Npe6 |
| | derivatives | |
| tin etioporphyrin (SnET2) | pheoboride-a | bacteriochlorophyll-a |
| naphthalocyanines | phthalocyanines | zinc phthalocyanines |
| Growth Factor Receptor | EGFR antagonists | HER-2 antagonists |
| Antagonists | | |

Examples of chemotherapeutic agents that are particularly useful in conjunction with radiosensitizers include, for example, camptothecin, carboplatin, cis-platin, daunorubicin, doxorubicin, interferon (alpha, beta, gamma), irinotecan, hydroxyurea, chlorambucil, 5-fluorouracil (5-FU), methotrexate, 2-chloroadenosine, fludarabine, azacytidine, gemcitabine, pemetrexed, interleukin 2, irinotecan, docetaxel, paclitaxel, topotecan, and therapeutically effective analogs and derivatives of the same.

In accordance with the present invention, compounds of the present invention are useful in combination with gemcitabine, or in combination with gemcitabine and paclitaxel. Compounds of the present invention also are useful in combination with pemetrexed, or in combination with pemetrexed and cisplatin, carboplatin, or other platins. A present Chk1 inhibitor also can be administered in combination with gemcitabine and pemetrexed.

A present Chk1 inhibitor administered in combination with gemcitabine can be useful in the treatment of, for example, pancreatic carcinoma, leiomyosarcoma of the uterus, bone sarcoma, metastatic nonsmall cell lung cancer, extremity and trunk soft tissue sarcoma, renal cell cancer, adenocarcinoma, and Hodgkin's disease. A present Chk1 inhibitor administered with pemetrexed can be useful in the treatment of mesothelioma.

As appreciated by persons skilled in the art, reference herein to treatment extends to prophylaxis, as well as to treatment of established diseases or symptoms. Reference to treatment also refers to the reduction of the rate of proliferation or the reduction of recurrence of the treated indication. It is further appreciated that the amount of a compound of the invention required for use in treatment varies with the nature of the condition being treated, and with the age and the condition of the patient, and is ultimately determined by the attendant physician or veterinarian.

In general, however, doses administered for adult human treatment typically are in the range of 0.001 mg/kg to about 100 mg/kg per day. The desired dose can be conveniently administered in a single dose, or as multiple doses administered at appropriate intervals, for example as two, three, four or more subdoses per day. In practice, the physician determines the actual dosing regimen most suitable for an individual patient, and the dosage varies with the age, weight, and response of the particular patient. The above dosages are exemplary of the average case, but individual instances can exists wherein higher or lower dosages are merited, and such are within the scope of the present invention.

Contact of the cell population with a present Chk1 inhibitor can likewise occur at any dose and time sufficient to achieve substantial abrogation of the cell cycle checkpoint. Typically, though not necessarily, such times include up to about 72 to about 96 hours, depending upon various factors. In some embodiments, it is desirable or necessary to administer Chk1 inhibitor over a period of up to about several weeks or more, as determined by the attending physician or technician. Thus, a present Chk1 inhibitor typically can be administered for up to about 1 hour, up to about 2 hours, up to about 3 hours, up to about 4 hours, up to about 6 hours, up to about 12 hours, up to about 18 hours, up to about 24 hours, up to about 48 hours, or up to about 72 hours. Persons skilled in the art appreciate that the ranges of time expressed herein are merely exemplary and that ranges and subranges within and outside those expressed also are within the scope of the invention.

Chk1 inhibitors of the present invention can be administered over a plurality of doses. For example, the Chk1 inhibitor can be given at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

### EXAMPLES

### Example 1

### Determination of IC₅₀ Values of the Chk1 Inhibitors

Human Chk1 cDNA was identified and cloned as described previously in International Application Publication No. WO 99/11795, filed Sep. 4, 1998. A FLAG tag was inserted in frame with the amino terminus of the full-length Chk1. The 5′ primer contains an EcoRI site, a Kozak sequence, and also encodes a FLAG® tag for affinity purification using the M2 Antibody (Sigma, Saint Louis, Ill.). The 3′ primer contains a SalI site. The PCR-amplified fragment was cloned into pCI-Neo as an EcoRI-SalI fragment (Invitrogen, Carlsbad, Calif.), then subcloned as an EcoRI-NotI fragment into pFastBacI (Gibco-BRL, Bethesda, Md.). Recombinant baculovirus was prepared as described in the Gibco-BRL Bac-to-Bac manual and used to infect Sf-9 cells grown in CCM3 medium (HyClone Laboratories, Logan, Utah) for expression of FLAG®-tagged Chk1 protein.

FLAG®-tagged Chk1 was purified from frozen pellets of baculovirus-infected SF9 cells. Frozen cell pellets were mixed with an equal volume of 2× lysis buffer containing 100 mM Tris-HCl pH 7.5, 200 mM NaCl, 50 mM B-glycerophosphate, 25 mM NaF, 4 mM MgCl₂, 0.5 mM EGTA, 0.2% TWEEN®-20, 2 mM sodium vanadate, 2 mM DTT, and a cocktail of protease inhibitors (Complete mini, Boehringer Mannheim 2000 catalog #1836170). Cells then were dounced 20 times with the loose pestle of a dounce homogenizer and centrifuged at 48,400×g for 1 hour. The M2 affinity was prewashed with 10 column volumes of 50 mM glycine pH 3.5 followed by 20 mM Tris pH 7.5, 150 mM NaCl alternating three times and ending with a Tris NaCl wash. The column then was washed with 25 column volumes of 20 mM Tris pH 7.5, 150 mM NaCl, 0.1% TWEEN®-20, 1 mM EGTA, 1 mM EDTA and 1× complete mini protease tablets. The cleared lysate then was bound to M2 affinity resin in batch at 4° C. for 4 hours. The mixture of resin and lysate then was poured into a column and the flow through collected. The resin was washed with 10 column volumes of 20 mM Tris pH 7.5, 150 mM NaCl, and 3 mM N-octyl glucoside. FLAG®-tagged Chk1 then was eluted from the column with 6 column volumes of cold 20 mM Tris pH 7.5, 150 mM NaCl, 3 mM N-octyl glucoside containing 0.5 mg/mL FLAG® peptide (Sigma, 2000 Catalog #F-3290). Three fractions were collected an analyzed for the presence of FLAG-tagged Chk1.

The protein kinase was used in an assay for Chk1 kinase activity that includes 100 ng purified FLAG®-Chk1(150 pmol of ATP/min), 20 μm Cdc25C peptide (H-leutyr-arg-ser-pro-ser-met-pro-glu-asn-leu-asn-arg-arg-argarg-OH) (SEQ ID NO: 1), 4 μm ATP, 2 μCi [³²P] γ-ATP, 20 mM Hepes pH 7.2, 5 mM MgCl₂, 0.1% NP40, and 1 mM DTT. This assay was used to determine IC₅₀ of compounds of the present invention. Reactions were initiated by the addition of ATP-containing reaction mix and carried out at room temperature for 10 min. Reactions were stopped by the addition of phosphoric acid (150 mM final concentration) and transferred to phosphocellulose discs. The phosphocellulose discs were washed five times with 150 mM phosphoric acid and air-dried. Scintillation fluid was added and discs were counted in a Wallac scintillation counter. Chk1 inhibitors of the present invention that were sub-jected to the assay have measured IC₅₀ values of about 8 to about 500 nM.

### Example 2

### Selectivity

Chk1 inhibitors of the present invention were tested for selectivity as against one or more other protein kinases, i.e., DNA-PK, Cdc2, Casein Kinase I (CKI), Chk2, p38 MAP kinase, ERK kinase, Protein Kinase A (PKA), and/or calcium-calmodulin protein kinase II (CaM KII).

Assay procedures for all of these kinases except Chk2 have been previously described in the literature, including U.S. Patent Publication No. 2002-016521 A1, and U.S. patent application Ser. No. 08/184,605, filed Jan. 21, 1994, both of which are herein incorporated by reference.

Activity of the compounds against Chk2 was assayed as follows: 128 ng of purified His-tagged Chk2 was incubated with up to 100 mM Chk1 inhibitor in the presence of 4 mM ATP, 1 mCi [³²P] γ-ATP, 20 mM Hepes pH 7.5, 5 mM MgCl₂, and 0.25% NP40 for 20 minutes at room temperature. Reactions were stopped with a final concentration of 150 mM phosphoric acid, and ⅝ of the reaction mixture was transferred to phosphocellulose discs. The discs were washed five times with 150 mM phosphoric acid, and air-dried. Scintillant was added and radioactivity was counted using a Wallac beta counter.

p38 MAP kinase, ERK kinase, PKA, CaM KII, and Cdc2 were purchased from New England Biolabs, and assays were performed according to the manufacturer's instructions using 4-50 μM ATP and testing Chk1 inhibitor concentrations as high as 100 μM. All inhibitors tested showed at least a 100-fold selectivity for Chk1 over the other enzymes.

### Example 3

### Chk1 Inhibitors of the Invention Inhibit Chk1 Function in Cells

To establish that the Chk1 inhibitors of the invention inhibit Chk1 function in-cells, inhibitors can be tested in molecular cell-based assays. Because mammalian Chk1 has been shown to phosphorylate Cdc25C in vitro, suggesting that it negatively regulates cyclin B/cdc2 in response to DNA damage, the ability of the Chk1 inhibitors to enhance the activity of CyclinB/cdc2 can be analyzed. The experiment can be designed as follows: HeLa cells are irradiated with 800 rads and incubated for 7 hours at 37° C. Because these cells are functionally p53 negative, they arrest exclusively in G2. Then, nocodazole is added to a concentration of 0.5 μg/mL and the cells are incubated for 15 hours at 37° C. The addition of nocodazole is designed to trap any cells that progress through the G2 arrest into M. Finally, a Chk1 inhibitor is added for 8 hours, the cells harvested, lysed and immunoprecipitated equal amounts of protein with an antibody to Cyclin Bl (New England Biolabs) as suggested by the manufacturer. Immunoprecipitates then are analyzed for Cyclin B-associated cdc2 kinase activity by assaying histone Hi kinase activity (Yu et al., J Biol. Chem., Dec. 11, 1998; 273(50):33455-64).

In addition, the ability of the subject Chk1 inhibitors to abrogate the ionizing radiation-induced G2 DNA damage checkpoint can be established using mitotic index assay experiments. HeLa cells (approximately 1×10⁶) are treated as described above. Cells are harvested by centrifugation, washed once with PBS, then resuspended in 2.5 mL of 75 mM KCl and centrifuged again. The cells then are fixed in 3 mL of freshly prepared cold acetic acid:methanol (1:3) and incubated on ice for 20 minutes. Cells are pelleted, fix solution aspirated and resuspended in 0.5 mL of PBS. Mitotic spreads are pre-pared by pipetting 100 μL of the fixed cells onto a glass microscope slide and flooding the sample with 1 mL of fix solution. Slides then are air dried, stained with Wright's stain (Sigma) for 1 minute, followed by one wash with water and one wash with 50% methanol. The presence of condensed chromosomes and lack of nuclear envelope identifies mitotic cells.

### Example 4

### Chk1 Inhibitors of the Present Invention Enhance Killing of Cells by Cancer Treatments

To demonstrate that the inhibition of Chk1 by a compound of the present invention sensitizes targeted cells to the killing effect of DNA-damaging agents, cells can be incubated in the presence of a present Chk1 inhibitor and exposed to either irradiation or a chemical DNA-damaging agent. Cells plated at a density of 1000-2000 per well in 96-well microtitre plates are grown in RMPI 1640 containing 10% FBS, 100 U/mL penicillin and 100 μg/mL streptomycin for 18 hours at 37° C. in a humidified incubator with 5% CO₂. Cells tested can include any cells or cell lines of interest, such as HeLa, ACHN, 786-0, HCT116, SW620, HT29, Colo205, SK-MEL-5, SK-MEL-28, A549, H322, OVCAR-3, SK-OV-3, MDA-MB-231, MCF-7, PC-3, HL-60, K562, and MOLT4. All cell line designations refer to the following human cell lines:

| | |
|---|---|
| HeLa | cervical adenocarcinoma |
| ACHN | renal adenocarcinoma |
| 786-0 | renal adenocarcinoma |
| HCT116 | colon carcinoma |
| SW620 | colon carcinoma, lymph node metastasis |
| HT-29 | colonrectal adenocarcinoma |
| Colo205 | colon adenocarcinoma |
| SK-MEL-5 | melanoma |
| SK-MEL-28 | malignant melanoma |
| A549 | lung carcinoma |
| H322 | broncholoalveolar carcinoma |
| OVCAR-3 | ovarian adenocarcinoma |
| SK-OV-3 | ovarian adenocarcinoma |
| MDA-MB-231 | breast adenocarcinoma |
| MCF-7 | breast adenocarcinoma |
| PC-3 | prostate adenocarcinoma, from metastasis |
| | to bone |
| HL-60 | acute promyelocytic leukemia |
| K562 | chronic myelogenous leukemia |
| MOLT4 | acute lymphoblastic leukemia; T |
| | lymphoblast |

Cells are treated with media containing chemotherapeutic drugs alone or chemotherapeutic drugs and a Chk1 inhibitor. Cells are incubated for approximately 5 days before growth is measured by determination of levels of ³H-thymidine uptake. Chemotherapeutic drugs include etoposide, doxorubicin, cis-platin, chlorambucil, 5-fluorouracil (5-FU). The drug concentration necessary to inhibit cell growth to 90% of untreated control cells is defined as the GI₉₀.

Compounds of the present invention can be tested with additional antimetabolites, including methotrexate, hydroxyurea, 2-chloroadenosine, fludarabine, azacytidine, and gemcitibine to assess therein ability to enhance killing of the agents. Compounds of the present invention can be compared to one another by assessing enhanced killing of HT29 colorectal carcinoma in combination with gemcitibine.

In addition, the ability of the Chk1 inhibitors of the invention to enhance killing by radiation can be tested.

### Example 5

### Animal Tumor Models

To test the ability of the Chk1 inhibitors of the invention to enhance the killing of tumors by DNA damaging agents in mice, xenograft tumor models using colon tumor cell lines are established. 5-fluorouracil (5-FU) or gemcitabine can be used as DNA damaging agents. HT29 and Colo205 (human colon carcinoma) and H460 and Calu-6 (nonsmall cell carcinoma) cells can be used to propagate xenograft tumors in 6-8 week old female thymic Balb/c (nu/nu) mice. Mice are maintained in a laminar airflow cabinet under pathogen-free conditions and fed sterile food and water ad libitum. Cell lines are grown to subconfluence in RPMI 1640 media supplemented with 10% FBS, 100 U/mL penicillin, 100 μg/mL streptomycin, and 1.5 mM L-glutamine in a 5% CO₂ humidified environment. Single cell suspensions are prepared in CMF-PBS, and cell concentration adjusted to 1×10⁸ cells/mL. Mice are inoculated subcutaneously (s.c.) on the right flank or right leg with a total of 1×10⁷ cells (100 μL).

Mice are randomized (5-15 mice/group) into four treatment groups and used when tumors reach a volume of 75-100 cm³ (usually 7-11 days post-inoculation). Tumors are measured with vermier calipers and tumor volumes are estimated using the empirically derived formula: tumor volume (cm³)=tumor length (cm) x tumor width (cm)×tumor depth (cm)/3.3. Treatment consists of i) 100 μL intraperitoneal (i.p) injection of gemcitabine at 160 mg/kg. A delay in tumor growth is observed in the mice treated with gemcitabine. Treatment of mice with both 160 mg/kg gemcitabine in combination with oral administration of Chk1 inhibitors is expected to reduce tumor volumes and prolong life. Tumor size is monitored every other day for the duration of the experiment.

Obviously, many modifications and variations of the invention as hereinbefore set forth can be made without departing from the spirit and scope thereof, and, therefore, only such limitations should be imposed as are indicated by the appended claims.

1.-28. (canceled)

29. A compound having a formula 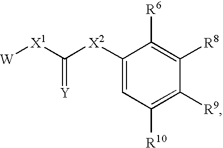
wherein X¹ and X² are —N(H)—;
Y is O;
W is pyrazinyl substituted with CF₃;
R⁶ is OR¹¹;
R⁸ and R⁹ are hydro;
R¹⁰ is CF₃ or CH₃;
R¹¹ is CH₃ substituted with piperidin-3-yl or 1-methyl-piperidin-3-yl;
or a pharmaceutically acceptable salt thereof.

30. A pharmaceutical composition comprising a compound of claim 29, or a pharmaceutically acceptable salt thereof, together with a pharmaceutically acceptable diluent or carrier.

31. A method of treating a cancer selected from the group consisting of a colorectal cancer, a head and neck cancer, a pancreatic cancer, a breast cancer, a gastric cancer, a bladder cancer, a vulvar cancer, a leukemia, a lymphoma, a melanoma, a renal cell carcinoma, an ovarian cancer, a brain tumor, an osteosarcoma, and a lung carcinoma which comprises administering to a patient in need thereof a therapeutically effective amount of a compound of claim 29 or a pharmaceutically acceptable salt thereof.

32. A compound selected from the group consisting of
1-[2-(piperidin-3-ylmethoxy)-5-trifluoromethyl-phenyl]-3-(5-trifluoromethyl-pyrazin-2-yl)-urea hydrochloride salt;
1-[5-methyl-2-(piperidin-3-ylmethoxy)-phenyl]-3-(5-trifluoromethyl-pyrazin-2-yl)-urea hydrochloride salt; and
1-[5-methyl-2-(1-methyl-piperidin-3-ylmethoxy)-phenyl]-3-(5-trifluoromethyl-pyrazin-2-yl)-urea.


SEQUENCE LISTING (1 sequence)

SEQ ID NO: 1 - protein, 16 aa, artificial
  synthetic peptide
LYRSPSMPENLNRRRR